(19) United States
(12) Patent Application Publication
(10) Pub. No.: US 2017/0327850 A1
(43) Pub. Date: Nov. 16, 2017

(54) ENGINEERED FUNGI FOR ITACONIC ACID PRODUCTION

(71) Applicant: BOARD OF REGENTS, THE UNIVERSITY OF TEXAS SYSTEM, Austin, TX (US)

(72) Inventors: Hal ALPER, Austin, TX (US); John BLAZECK, Austin, TX (US); Andrew HILL, Austin, TX (US)

(21) Appl. No.: 15/523,218

(22) PCT Filed: Oct. 29, 2015
(86) PCT No.: PCT/US2015/057968

§ 371 Date: Apr. 28, 2017

Related U.S. Application Data
(60) Provisional application No. 62/072,734, filed on Oct. 30, 2014.

(51) Int. Cl. C12P 7/44; C07K 14/39; C12N 9/88; C12N 9/12; C12N 9/10; C07K 14/47; C12N 15/81; C12N 9/78

(52) U.S. Cl. CPC C12P 7/44; C12Y 401/01006; C12Y 305/04006; C12Y 203/03001; C12N 15/815; C07K 14/39; C12N 9/78; C12N 9/1205; C12N 9/1025; C07K 14/47; C12N 9/88

(57) ABSTRACT

Genetically engineered oleaginous fungi (e.g., engineered Yarrowia lipolytica) are provided for use in itaconic acid production. In some aspects, the engineered fungi comprise a transgene for expression of a cis-aconitic acid decarboxylase (CAD) enzyme and, optionally, one or more further genetic modifications. Methods and culture systems for production of itaconic acid using such fungi are also provided.

This application claims the benefit of priority to U.S. Provisional Application Ser. No. 62/072,734, filed on Oct. 30, 2014, the entire contents of which are hereby incorporated by reference.

### BACKGROUND OF THE INVENTION

### 1. Field of the Invention

The present invention relates generally to the fields of genetic engineering and metabolic engineering. More particularly, it concerns engineered fungi that can be used for itaconic acid production and methods of using the same.

### 2. Description of Related Art

Manipulation of metabolic flux within microorganisms can enable the efficient and economical production of a variety of value-added chemicals. Application of metabolic engineering for the renewable production of biofuels and other chemical alternatives to petroleum derivatives is of particular interest. One such chemical is itaconic acid, which is naturally produced in several Aspergillus species and has the potential to replace traditionally petroleum-derived materials. Itaconic acid is a versatile monomer with various applications in plastics and rubber (Okabe et al., 2009; Tate, 1981; Tsai et al., 2000). Furthermore, polyitaconic acid can serve as an alternative to polyacrylic acid, a high volume commodity petrochemical (Itaconix, 2009; Nuss and Gardner, 2013). This utility has caused itaconic acid to be recognized as a top 12 value-added chemical from biomass by the Department of Energy in 2004 (Werpy and Petersen, 2004); however, expansion of the market for products derived from itaconic acid depends upon decreased production costs (Nuss and Gardner, 2013; Okabe et al., 2009).

The production of itaconic acid was first discovered in 1932 by the fungus Aspergillus itaconicus (Kinoshita, 1932) and has been detected in a variety of other species, including A. terreus (Okabe et al., 2009; Tevz et al., 2010). Metabolomics studies determined that itaconic acid production in A. terreus is achieved through the decarboxylation of the TCA cycle intermediate, cis-aconitic acid by the cis-aconitic acid decarboxylase (CAD) enzyme (Bonnarme et al., 1995; Kanamasa et al., 2008).

Current industrial production of itaconic acid is carried out in Aspergillus terreus fermentations (Tevz et al., 2010). Attempts to rationally engineer Aspergillus species for itaconic acid production have achieved modest success (Tevz et al., 2010; van der Straat et al., 2014); however, media optimization and mutagenesis have yielded far greater improvements (Hevekerl et al., 2014; Kautola et al., 1991; Li et al., 2012). Although high titers of itaconic acid have been achieved in A. terreus, the organism suffers from poor growth in media optimal for itaconic acid production (Okabe et al., 2009; Tevz et al., 2010). Furthermore, A. terreus is negatively affected by shear stress, precluding fermentations in conventional stirred-tank bioreactors (Okabe et al., 2009; Park et al., 1994; Yahiro et al., 1995). Accordingly, there remains a need for genetically engineered organisms that are easy to cultivate and have established track records for large and industrial scale cultivation, particularly for use in industrial scale biological production of itaconic acid.

### SUMMARY OF THE INVENTION

In a first embodiment the invention provides a transgenic oleaginous fungus, the fungus comprising at least a first transgenic nucleic acid molecule encoding a cis-aconitic acid decarboxylase (CAD) enzyme operably linked to a promoter functional in the fungus. In some aspects, the nucleic acid encoding the CAD enzyme is integrated into the genome of the fungus and/or is present as an episomal genetic element. In further aspects, a transgenic fungus of the embodiments further comprises at least a second genetic modification that increases expression or activity of a gene product selected from the group consisting of AMP deaminase (AMPD), iron-regulatory protein, aconitase, citrate synthase, small acid resistance transporter, citrate transport protein and phosphofructokinase (e.g., one or more of the gene products provided in Table 3). In some aspects, the oleaginous fungus is Yarrowia lipolytica (e.g., a Y. lipolytica strain). In certain aspects, the fungus may have been adapted to low pH growth conditions (to reduce salt formation in the industrial scale fermentation).

In some aspects, a fungus of the embodiments comprises a transgene that is integrated into the fungal genome. In further aspects, a transgene may be comprised in an episomal genetic element. For example, a transgenic fungus may comprise a genome integrated or an episomal nucleic acid molecule encoding a CAD enzyme operably linked to a promoter functional in the fungus. In other aspects, the fungus comprises both a genome integrated and an episomal nucleic acid molecule each encoding a CAD enzyme operably linked to a promoter functional in the fungus. In certain aspects, the CAD enzyme may be an Aspergillus terreus CAD enzyme (Gene ID AB326105).

In further aspects, a transgenic fungus of the embodiments includes a genetic modification comprising an expressible transgene encoding a gene product selected from the group consisting of AMPD, iron-regulatory protein, aconitase, citrate synthase, small acid resistance transporter, citrate transport protein and phosphofructokinase. In another aspect, the modification comprises promoter mutation or replacement of a promoter linked to an AMPD, iron-regulatory protein, aconitase, citrate synthase, small acid resistance transporter, citrate transport protein or phosphofructokinase gene in the fungus (e.g., thereby increasing the expression of the gene compared to a wild type fungus). In a further aspect, the genetic modification comprises mutation of a coding sequence for an AMPD, iron-regulatory protein, aconitase, citrate synthase, small acid resistance transporter, citrate transport protein or phosphofructokinase gene that increases activity of the gene product. In some aspects, a transgenic fungus genetic modifications of at least 2, 3, 4, 5, 6 or more genes that increases expression or activity of a gene product (e.g., such as genes encoding AMPD, iron-regulatory protein, aconitase, citrate synthase, small acid resistance transporter, citrate transport protein and/or phosphofructokinase). In still further aspects, the fungus further comprises a transgene encoding a selectable (e.g., a drug selection marker) or screenable marker.

Thus, in some aspects, a transgenic fungus comprises a genetic modification for overexpression or increased activity of an iron-regulatory protein. For example, the fungus may comprise a transgene encoding an iron-regulatory protein, such as the iron-regulatory protein of O. cuniculus iron-regulatory protein (Gen ID Q01059). Additionally or alternatively, the iron-regulatory protein may comprise a S711D mutation relative to the wild type protein.

In further aspects, a transgenic fungus comprises a genetic modification for overexpression or increased activity of a small acid resistance transporter protein. For example, the fungus may comprise a transgene encoding a small acid resistance transporter protein, such as the small acid resistance transporter of Y. lipolytica (Gen ID YALI0E10483g).

In yet further aspects, a transgenic fungus comprises a genetic modification for overexpression or increased activity of a citrate transport protein. For example, the fungus may comprise a transgene encoding a citrate transport protein, such as the citrate transport protein of Y. lipolytica (Gen ID YALI0F26323g).

In yet still further aspects, a transgenic fungus comprises a genetic modification for overexpression or increased activity of aconitase. For example, the fungus may comprise a transgene encoding an aconitase protein, such as the aconitase of Y. lipolytica aconitase (Gen ID YALI0D09361g). In some cases, the aconitase may not include a mitochondrial localization signal (MLS).

In certain aspects, a transgenic fungus comprises a genetic modification for overexpression or increased activity of a citrate synthase. For example, the fungus may comprise a transgene encoding a citrate synthase protein, such as the citrate synthase of Y. lipolytica (Gen ID YALI0E02684g). In some cases, the citrate synthase may not include a MLS.

In further aspects, a transgenic fungus comprises a genetic modification for overexpression or increased activity of a phosphofructokinase. For example, the fungus may comprise a transgene encoding a phosphofructokinase protein, such as the phosphofructokinase of Y. lipolytica (Gen ID YALI0D16357g). Alternatively or additionally, a phosphofructokinase may comprise a K731A or K731R mutation relative to the wild type protein (a mutation to reduce feedback inhibition).

In still further aspects, a transgenic fungus comprises a genetic modification for overexpression or increased activity of an AMPD enzyme. For example, the fungus may comprise a transgene encoding an AMPD enzyme, such as the AMPD enzyme of Y. lipolytica AMPD enzyme (Gene ID YALI0E11495g). In some aspects, the transgenic nucleic acid molecule encoding the AMPD enzyme may be integrated in the Y. lipolytica genome or may be comprised in an UAS1B16-TEF expression cassette.

In a further embodiment there is provided a culture system comprising a population of transgenic oleaginous fungi of the embodiments and a growth medium. In some aspects the culture may comprise itaconic acid. Media for components for use according the embodiments are well known in the art and further detailed herein below. In certain aspects, however, the media may comprise carbon (e.g., glucose) and nitrogen (e.g., ammonium) sources, said carbon and nitrogen sources present in a molar ratio of at least 30 (mol C:mol N). In some aspects, said carbon and nitrogen sources are present in a ratio of between about 30 and 100; 30 and 80; 30 and 600; 100 and 500; 200 and 500; or 300 and 500 (mol C:mol N). In still further aspects, the medium is not supplemented with amino acids. In certain aspects a culture system of the embodiments may be comprised in a shaker flask or a bioreactor.

In yet a further embodiment, there is provided a method for producing an organic commodity chemical comprising culturing transgenic oleaginous fungi according to the embodiments in a growth media and collecting the organic commodity chemical from the fungus and/or the growth media. In some aspects, the commodity chemical may comprise itaconic acid. In certain cases, culturing of the fungi may be in shaker flask or in a bioreactor. In some aspects, the culture may be in a batch, fed-batch, or a continuous feed system.

In a particular aspect, the culturing is in a bioreactor and the transgenic oleaginous fungi comprises a transgenic nucleic acid molecule encoding an aconitase operably linked to a promoter functional in the fungus, wherein the aconitase does not include a MLS, and comprising a genome integrated and an episomal nucleic acid molecule each encoding a CAD enzyme operably linked to a promoter functional in the fungus.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Itaconic acid production in Y. lipolytica with episomal CAD expression. Episomal expression of the CAD enzyme, driven by the UAS1B16-TEF promoter, in Y. lipolytica PO1f enabled itaconic acid production of 33 mg/L. CAD expression in a Y. lipolytica strain engineered to constitutively express AMPD increased itaconic acid production to 159 mg/L. Strains were cultivated in C₂₀N_{1.365} media with amino acid supplementation for four days. Error bars represent the standard deviation of biological triplicates.

FIGS. 2A-2B: Altering C:N ration to increase organic acid production. (A) PO1f and PO1f AMPD overexpression backgrounds, harboring episomal CAD expression cassettes, were analyzed for itaconic acid production when cultivated in three media formulations for four days, C₂₀N_{1.365}, C₂₀N_{0.055}, and C₈₀N_{1.365}, where C and N represent g/L glucose and g/L ammonium, respectively. Increasing C:N ratio, by decreasing nitrogen level or increasing glucose level, effectively increased itaconic acid production in PO1f. No effect on itaconic acid production was seen in the AMPD expression background. (A) Interestingly, the C₂₀N_{0.055} formulation stimulated exceedingly high citric acid production in the AMPD expression background. Other media formulation did not stimulate citric acid accumulation. Error bars represent standard deviations of biological triplicates.

FIG. 3: Chromosomally expressing CAD and eliminating amino acid supplementation increase itaconic acid production. PO1f and PO1f AMPD overexpression backgrounds, harboring chromosomal CAD expression cassettes, were assayed for itaconic acid production after a four day cultivation in standard C₂₀N_{1.365} media (including CSM amino acid supplementation). Chromosomal CAD expression increased itaconic acid titers to 136 mg/L and 226 mg/L for the PO1f and PO1f AMPD backgrounds, respectively. Cultivation in C₂₀N_{1.365} minimal media without amino acid supplementation increased itaconic acid production to 272 mg/L in the PO1f AMPD CAD strain. Error bars represent the standard deviation of biological triplicates.

FIGS. 4A-4D: Time course of itaconic acid production. PO1f and PO1f AMPD overexpression backgrounds, harboring chromosomal CAD expression cassettes, were assayed for itaconic acid production after a two, three, four, six and seven days cultivation in minimal (A) C₂₀N_{1.365} and (B) C₂₀N_{0.055} media. Increasing cultivation duration increased itaconic acid production to 365 mg/L for PO1f CAD and 336 mg/L for PO1f AMPD CAD. (C) Citric acid accumulation in the minimal C₂₀N_{0.055} media reaches 437 mg/L for PO1f AMPD CAD and 157 mg/L for PO1f CAD, but was not detectable in minimal C₂₀N_{1.365} media. (D) OD₆₀₀ measurements indicate that PO1f CAD and PO1f AMPD CAD reached peak cell density after 3-4 days. Error bars represent the standard deviation of biological triplicates.

FIGS. 5A-5B: Fine-tuning nitrogen depletion. (A) The PO1f AMPD CAD chromosomal expression strain was cultivated for seven days in C₂₀N_{1.365}, C₂₀N_{0.273}, and C₂₀N_{0.1365} minimal media and assayed for itaconic acid production. Decreasing nitrogen content by 80% with the C₂₀N_{0.273} resulted in an increased itaconic acid titer to 667 mg/L, while a 90% nitrogen reduction with C₂₀N_{0.1365} media decreased itaconic acid titer. Error bars represent the standard deviation of biological triplicates. (B) Further media optimization for itaconic acid production. Test was conducted with the AMPD, CAD strain with 20 g/L at varying ammonium concentrations.

FIGS. 6A-6C: Strain engineering for itaconic acid production tested in flask scale fermentations. (A) Media formulation was 20 g/L glucose and 6.7 g/L YNB without amino acids. Samples were tested after three days of growth. (B) Media formulation was 20 g/L glucose, 1.34 g/L YNB without amino acids, and 1.36 g/L YNB without amino acids and ammonium sulfate. The * indicates evolved PO1F strain for pH tolerance (pH 2.8) with two copies CAD integrated. (C) Media formulation was 20 g/L glucose, 1.34 g/L YNB without amino acids, and 1.36 g/L YNB without amino acids and ammonium sulfate. Samples were tested after seven days of growth.

FIGS. 7A-7B: Bioreactor fermentations of itaconic acid producting strains. Fermentations were carried out in 80 g/L glucose and 6.7 g/L YNB without amino acids. Controlled settings were: temperature (28° C.), flow rate (2.5 vvm), % DO (50%), agitation (250-800 RPM), and pH (5.0). pH was adjusted using base control with 2.5 M sodium hydroxide. The * indicates the fermentation was conducted at a pH of 3.5 instead of 5.0.

FIGS. 8A-8B: Bioreactor fermentation of AMPD CAD strain in 1.5 L bioreactor fermentation. Fermentation was carried out in 80 g/L glucose and 6.7 g/L YNB without amino acids. Controlled settings were: temperature (28° C.), flow rate (2.5 vvm), % DO (50%), agitation (250-800 RPM), and pH (3.5 in A and 5.0 in B) in. pH was adjusted using base control with 2.5 M sodium hydroxide.

FIGS. 9A-9B: Bioreactor fermentation of AMPD CAD strain in 1.5 L bioreactor fermentation. (A) Fermentation was carried out in 40 g/L glucose and 3.35 g/L YNB without amino acids initially. After the third day, the media was subjected to 20 g/L glucose spikes every 24 hours until the sixth day for a final supplied glucose concentration of 120 g/L. Controlled settings were: temperature (28° C.), flow rate (2.5 vvm), % DO (50%), agitation (250-800 RPM), and pH (5.0). pH was adjusted using base control with 2.5 M sodium hydroxide. (B) Fermentation was carried out in 120 g/L glucose and 3.35 g/L YNB without amino acids. Controlled settings were: temperature (28° C.), flow rate (2.5 vvm), % DO (50%), agitation (250-800 RPM), and pH (5.0). pH was adjusted using base control with 2.5 M sodium hydroxide.

FIGS. 10A-10D: Bioreactor fermentation of (A) S2 CAD, ACONOMLS epi, (B) S1,S2 CAD strain, (C) CAD, CAD epi, AMPD epi, strain, and (D) S1,S2 CAD, ACONOMLS epi, CAD epi strain. Fermentation was carried out in 80 g/L glucose and 6.7 g/L YNB without amino acids. Controlled settings were: temperature (28° C.), flow rate (2.5 vvm), % DO (50%), agitation (250-800 RPM), and pH (3.5 in A; 5.0 in B and C). pH was adjusted using base control with 2.5 M sodium hydroxide.

FIGS. 11A-11C: pH tolerance fermentations. Cells were inoculated to an initial OD of 0.01. (A) pH Tolerance fermentation for PO1F strain with media containing 20 g/L glucose, 0.79 g/L CSM, and 6.7 g/L YNB adjusted to various initial pH conditions. (B) pH Tolerance fermentation for native S1,S2 CAD strain with media containing 20 g/L glucose, 0.67 g/L CSM-LEU,-URA, and 6.7 g/L YNB adjusted to various initial pH conditions. (C) pH Tolerance fermentation for native AMPD, CAD, CAD epi, ACONOMLS epi strain with media containing 20 g/L glucose, 0.67 g/L CSM-LEU,-URA, and 6.7 g/L YNB adjusted to various initial pH conditions.

FIGS. 12A-12C: pH tolerance fermentations. Cells were inoculated to an initial OD of 0.01. (A) pH Tolerance fermentation for PO1F strain evolved for pH tolerance (3.4) with media containing 20 g/L glucose, 0.79 g/L CSM, and 6.7 g/L YNB adjusted to various initial pH conditions. (B) pH Tolerance fermentation for S1,S2 CAD evolved for pH tolerance (2.8) with media containing 20 g/L glucose, 0.67 g/L CSM-LEU,-URA, and 6.7 g/L YNB adjusted to various initial pH conditions. (C) pH Tolerance fermentation for AMPD, CAD, CAD epi, ACONOMLS evolved for pH tolerance (2.8) with media containing 20 g/L glucose, 0.67 g/L CSM-LEU,-URA, and 6.7 g/L YNB adjusted to various initial pH conditions.

FIGS. 13A-13C: pH tolerance fermentations. Cells were inoculated to an initial OD of 0.01. (A) pH Tolerance fermentation for native PO1F and a strain evolved pH tolerance (3.4) with media containing 20 g/L glucose, 0.67 g/L CSM-LEU,-URA, and 6.7 g/L YNB adjusted to an initial pH of 3.0. (B) pH Tolerance fermentation for native S1, S2 CAD and strains evolved pH tolerance (3.4, 2.8) with media containing 20 g/L glucose, 0.67 g/L CSM-LEU,-URA, and 6.7 g/L YNB adjusted to an initial pH of 3.0. (C) pH Tolerance fermentation for native AMPD, CAD, CAD epi, ACONOMLS and strains evolved pH tolerance (3.4, 2.8) with media containing 20 g/L glucose, 0.67 g/L CSM-LEU,-URA, and 6.7 g/L YNB adjusted to an initial pH of 3.0.

FIG. 14: Itaconic acid production test for strains evolved for pH tolerance in flask scale fermentations. Media formulation was 20 g/L glucose, 1.34 g/L YNB without amino acids, and 1.36 g/L YNB without amino acids and ammonium sulfate.

### DESCRIPTION OF ILLUSTRATIVE EMBODIMENTS

### I. The Present Invention

Y. lipolytica has the capacity to accumulate lipid content and organic acids through interrelated mechanisms (Papanikolaou, S. et al., 2009). While fatty acid accumulation requires an inhibition and reversal of TCA cycle flux to supply acetyl-CoA fatty acid precursor, organic acid accumulation requires only TCA cycle inhibition. In this manner, organic acid intermediates are accumulated, predominantly as citric and isocitric acid. The inventors have attempted to control TCA cycle inhibition in order to utilize these organic acid reserves for the production of itaconic acid, a value-added chemical monomer with diverse applications.

As detailed in the studies herein, it was surprisingly found that when the CAD enzyme (e.g., from A. terreus) was overexpressed in Y. lipolytica significant levels of itaconic acid could be produced tapping into the pool of citric, cis-aconitic, and isocitric acid reserves. Significant increases in the production of itaconic acid in Y. lipolytica could be achieved through the episomal expression of a CAD (cis-aconitic acid decarboxylase) enzyme. However, the inventors further increased itaconic acid by chromosomally expressing the CAD gene (either alone or in conjunction with episomal expression), thus avoiding the “half on/half off” phenotype observed in centromeric Y. lipolytica plasmids. Furthermore, by introducing additional genetic modifications into the engineered fungi the production of itaconic acid could be further enhanced. For example, overexpression of AMP deaminase resulted in significant increases in production. Likewise, overexpression or elevated activation of the gene products of Table 3 may result in yet further enhancements of itaconic acid.

The inventors also investigated alterations in the media conditions that favored itaconic acid production. In particular, it was found that by balancing the levels of carbon and nitrogen sources in the media the output of the system could be greatly enhanced. In particular, moderate nitrogen starvation conditions were found to be the most favorable for itaconic acid production. The additional use of a minimal media formulation, lacking amino acid supplementation, was found to yet further enhance production. In view of the resistance of Y. lipolytica to shear stress bioreactor culture of engineered organisms was also tested and found to likewise produce significant levels of itaconic acid. Thus, embodiments of the invention address a significant need in the art by providing genetically engineered oleaginous fungi that are suitable for industrial scale culture and able to produce high levels of itaconic acid.

### II. Oleaginous Fungi

A wide range of oleaginous fungi can be engineered in accordance with the current embodiments to provide biological systems for itaconic acid production. For example, in some aspects, the engineered organism may be Apiotrichum curvatum, Candida apicola, Candida curvata, Candida revkaufi, Candida pulcherrima, Candida tropicalis, Candida utilis, Cryptococcus curvatus, Cryptococcus terricolus, Debaromyces hansenii, Endomycopsis vernalis, Geotrichum carabidarum, Geotrichum cucujoidarum, Geotrichum histeridarum, Geotrichum silvicola, Geotrichum vulgare, Hyphopichia burtonii, Lipomyces lipoferus, Lipomyces lipofer, Lypomyces orentalis, Lipomyces starkeyi, Lipomyces tetrasporous, Pichia mexicana, Rodosporidium sphaerocarpum, Rhodosporidium toruloides, Rhodotorula aurantiaca, Rhodotorula dairenensis, Rhodotorula diffluens, Rhodotorula glutinus, Rhodotorula glutinis var. glutinis, Rhodotorula gracilis, Rhodotorula graminis, Rhodotorula minuta, Rhodotorula mucilaginosa, Rhodotorula mucilaginosa Rhodotorula mucilaginosa, Rhodotorula terpenoidalis, Rhodotorula toruloides, Sporobolomyces alborubescens, Starmerella bombicola, Torulaspora delbruekii, Torulaspora pretoriensis, Trichosporon behrend, Trichosporon brassicae, Trichosporon cutaneum, Trichosporon domesticum, Trichosporon fermentans, Trichosporon laibachii, Trichosporon loubieri, Trichosporon loubieri var. loubieri, Trichosporon montevideense, Trichosporon pullulans, Wickerhamomyces canadensis, Yarrowia lipolytica, or Zygoascus meyerae.

In some aspects, the engineered fungus is Yarrowia lipolytica. Y. lipolytica is a well-studied oleaginous yeast organism with well-developed tools for rational genetic engineering and has gained recognition for use in metabolic engineering applications (Barth and Gaillardin, 1996; Beopoulos et al., 2008; Blazeck, 2014; Blazeck et al., 2013a; Blazeck et al., 2011; Blazeck et al., 2013c; Fickers et al., 2003; Gon et al., 2014; Juretzek et al., 2001; Madzak et al., 2004, each incorporated herein by reference). In some aspects, a strain of Y. lipolytica for use according to the embodiments is a leucine and uracil auxotroph strain and/or is devoid of secreted protease activity. For example, the strain can be the PO1f strain (available from the ATCC # MYA-2613).

### III. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Itaconic Acid Production with Genetically Engineered Yarrowia lipolytica

Materials and Methods

Strains and Media for Routine Cultivations

Y. lipolytica expression vectors were propagated in Escherichia coli DH10B. E. coli DH10B was routinely cultivated in LB Media Broth (Teknova) supplemented with 50 μg/ml ampicillin for plasmid propagation at 37° C. with constant shaking. Yarrowia lipolytica strain PO1f (ATCC # MYA-2613), a leucine and uracil auxotroph devoid of any secreted protease activity (Madzak, C. et al., 2000, incorporated herein by reference) was used as the starting point for all strain construction Y. lipolytica studies.

YSC media consisted of 20 g/L glucose (Fisher Scientific), 0.79 g/L CSM supplement (MP Biomedicals), and 6.7 g/L Yeast Nitrogen Base w/o amino acids (Becton, Dickinson, and Company). YSC-URA, YSC-LEU, and YSC-LEU-URA media contained 0.77 g/L CSM-Uracil, 0.69 g/L CSM-Leucine, or 0.67 g/L CSM-Leucine-Uracil in place of CSM, respectively. YPD media contained 10 g/L yeast extract (Fisher Scientific), 20 g/L peptone (Fisher Scientific) and 20 g/L glucose, and was supplemented with 300 μg/ml Hygromycin B (Invitrogen) when Y. lipolytica necessary. S. cerevisiae BY4741 (MATa; his3Δ1; leu2Δ0; met15Δ0; ura3Δ0) obtained from EUROSCARF, Frankfurt, Germany was utilized for homologous recombination media construction of the CAD gene (described below) and was cultivated in YPD or the appropriate selection media.

Cloning Procedures

All restriction enzymes were purchased from New England Biolabs and all digestions were performed according to standard protocols. PCR reactions were set up with recommended conditions using Phusion high fidelity DNA polymerase (Finnzymes). Ligation reactions were performed overnight at room temperature using T4 DNA Ligase (Fermentas). Gel extractions were performed using the Fermentas GeneJET extraction kit purchased from Fisher ThermoScientific. E. coli minipreps were performed using the Zyppy Plasmid Miniprep Kit (Zymo Research Corporation). S. cerevisiae plasmid minipreps were performed using Zymoprep Yeast Plasmid Miniprep II kit (Zymo Research Corporation). E. coli maxipreps were performed using the Qiagen HiSpeed Plasmid Maxi Kit. Transformation of E. coli strains was performed using standard electroporator protocols (Sambrook and Russell, 2001). Large amounts of linearized DNA (>20 μg), necessary for Y. lipolytica PO1f transformation were cleaned and precipitated using a standard phenol:chloroform extraction followed by ethanol precipitation.

Genomic DNA (gDNA) was extracted from Y. lipolytica using the Wizard Genomic DNA Purification kit (Promega). Transformation of Y. lipolytica with episomal expression plasmids was performed using the Zymogen Frozen EZ Yeast Transformation Kit II (Zymo Research Corporation), with plating on appropriate selection plates. Transformation of Y. lipolytica PO1f with linearized cassettes was performed as described previously (Blazeck, J. et al., 2013). Briefly, PO1f and its derivatives were inoculated from glycerol stock directly into 10 mL YPD media, grown overnight, and harvested at an OD₆₀₀ between 9 and 15 by centrifugation at 1000×g for 5 minutes. Cells were washed in 8.0 mL TE buffer (10 mM Tris, 1 mM EDTA, pH=7.5), spun down, and resuspended in 8.0 mL TE buffer. 10⁸ cells were dispensed into separate microcentrifuge tubes for each transformation, spun down, and resuspended in 1.0 mL LiOAc buffer (100 mM LiOAc, adjusted to pH=6.0 with 2 M acetic acid). Cells were incubated with shaking at 30° C. for 60 minutes, spun down, resuspended in 90 μL LiOAc buffer, and placed on ice. 1-5 μg of linearized DNA was added to each transformation mixture in a total volume of 10 μL, followed by 25 μL of 50 mg/mL boiled salmon sperm DNA (Sigma-Aldrich). Cells were incubated at 30° C. for 15 minutes with shaking, before adding 720 μL PEG buffer (50% PEG8000, 100 mM LiOAc, pH=6.0) and 45 μL 2 M dithiothreitol. Cells were incubated at 30° C. with shaking at 225 rpm for 60 minutes, heat shocked for 10 minutes in a 39° C. water bath, spun down and resuspended in 1 mL sterile water. 200 μL of cells were plated on appropriate selection plates. All auxotrophic or antibiotic selection markers for genomic integrations were flanked with LoxP sites to allow for retrieval of integrated markers with the pMCS-UAS1B₁₆-TEF-Cre or pMCS-HYG-UAS1B₁₆-TEF-Cre replicative vectors (Blazeck et al., 2013a).

Plasmid Construction

Primer sequences can be found in Table 1 below. Four gBlocks gene fragments (Integrated DNA Technologies) were designed to encompass the intronless CAD gene sequence from Aspergillus terreus with at least 50 nucleotides overlapping between each gBlock and with the p416-UAS_{TEF}-UAS_{CIT}-UAS_{CLB}-P_{GPD} vector backbone (Kanamasa, S. et al., 2008; Blazeck, J. et al. 2012). Primers JB931/932 (SEQ ID NOs: 3/4), JB933/934 (SEQ ID NOs: 5/6), JB935/936 (SEQ ID NOs: 7/8), and JB937/938 (SEQ ID NOs: 9/10) were used to PCR amplify the four gBlocks. Amplified gBlock DNA fragments and linearized p416-UAS_{TEF}-UAS_{CIT}-UAS_{CLB}-P_{GPD} vector backbone were transformed into S. cerevisiae BY4741 following Hegemann's yeast transformation protocol (Guldener, U. et al., 1996) to enable homologous recombination mediated gene assembly (Shao, Z. et al., 2009). Plasmid p416-UAS_{TEF}-UAS_{CLB}-UAS_{CLB}-P_{GPD}-AtCAD was isolated from transformed BY4741 with a yeast miniprep, transformed into E. coli, miniprepped, and sequence confirmed.

TABLE 1
Primer sequences used in plasmid construction.

| | | SEQ |
|---|---|---|
| Primers | Sequence (5′-->3′) | ID NO: |
| JB865 | ggaacggtagatctcgagcgtcccaaaaccttctc | 1 |
| JB883 | gtggacgggccggcgtttggcgcccgttttttcg | 2 |
| JB931 | gtattgattgtaattctgtaaatctatttc | 3 |
| JB932 | cttgctgcaaagaccgcaggaaggacaatgcttgcagagtgtagggggg | 4 |
| | cttcgctgtgg | |
| JB933 | tttcatacaggctacggagcttgacgactaccacagcgaagccccccta | 5 |
| | cactctgcaag | |
| JB934 | gaggctctctgccgttgccc | 6 |
| JB935 | ttcttgggggactgttggcc | 7 |
| JB936 | agatgaagtaaccttcctggccagatc | 8 |
| JB937 | ccgtccagctggtcgaccag | 9 |
| JB938 | ctccttccttttcggttagagcggatgtggggggagggcgtgaatgtaa | 10 |
| JB1140 | gagtggcgcgccatgatttctgctattcgtccc | 11 |
| JB1141 | gcacttaattaattagagcttgaggccaacga | 12 |
| JB1142 | gagtggcgcgccatgcttaaggagcgattcgcc | 13 |
| JB1143 | gagtggcgcgccatgctggcttctcgagtttc | 14 |
| JB1144 | gcacttaattaattatttcttggaggcagcc | 15 |
| JB1145 | gagtggcgcgccatggccaacaacttcctcaacttc | 16 |
| JB1050 | gagtggcgcgccatgaccaaacaatctgcgg | 17 |
| JB1051 | gcacttaattaattataccagtggcgatttca | 18 |
| JB1168 | gagtggcgcgccatgtctaatccttttgcatacttag | 19 |
| JB1169 | gcacttaattaactactttgccatttttctaatca | 20 |
| LQ71 | aactctagatatgtctgataaaag | 21 |
| LQ72 | ttagcggccgcatactactgtatattc | 22 |
| LQ73 | aacgcggccgcctgcagactaaattta | 23 |
| LQ74 | ttcagatctctaacagttaatcttc | 24 |
| LQ311 | ACTGGGCGCGCCATGATTGAAGGAATCTCCTTTGCG | 25 |
| LQ312 | ACTGTTAATTAACTAACAAGGATCAATAATACCCTGCTC | 26 |
| LQ317 | ACGTGCTCGCGACGTCTGCTTCTGCCA | 27 |
| LQ318 | TGGCAGAAGCAGACGTCGCGAGCACGT | 28 |
| LQ319 | GACGTGCTCCGGACGTCTGCTTCTGCCA | 29 |
| LQ320 | TGGCAGAAGCAGACGTCCGGAGCACGTC | 30 |
| AH115 | GACTGGCGCGCCATGAGAGCCCTTCTGAACAAG | 31 |
| AH116 | GTCCTTAATTAATCATCTCATCATTCGTCGGAC | 32 |
| AH117 | GCCGAACCTTGGAAGTCCCT | 33 |
| AH118 | GTCCTTAATTAACTAAAGAATCTCCATGATCTTCTCATAGATGGT | 34 |
| AH119 | GACT GGCGCGCCATGGTTTCATCAGATACCAAGAAG | 35 |
| | GCCGAACCTTGGAAGTCCCT | |

Primers JB1050/1051 (SEQ ID NOs: 17/18) were used to amplify the A. terreus CAD gene from plasmid p416-UAS_{TEF}-UAS_{cIT}-UAS_{CLB}-P_{GPD}-AtCAD and insert it into the pUC-S2-UAS1B₁₆-TEF (Blazeck, J. et al., 2013a) and pMCS-UAS1B₁₆-TEF (Blazeck, J. et al., 2011) chromosomal and episomal expression vectors (respectively) with an AscI/PacI digest to form plasmids pUC-52-UAS1B₁₆-TEF-CAD and pMCS-UAS1B₁₆-TEF-CAD.

Primers LQ71/LQ72 (SEQ ID NOs: 21/22) were used to amplify ORI1001 from plasmid pMCS-Cen1 (Blazeck, J. et al., 2011) and insert it into plasmid pMCS-TEF-hrGFP (Blazeck, J. et al., 2011) with an XbaI/NotI-HF digest (replacing an identical ORI1001) to form plasmid pMCS-TEF-hrGFP-mod. Primers LQ73/LQ74 (SEQ ID NOs: 23/24) were used to amplify Ura3d1 from plasmid the pUC-S1-UAS1B₁₆-TEF (Blazeck, J. et al., 2013a) and insert it into plasmid pMCS-TEF-hrGFP-mod with an NotI-HF/BglII digest (replacing the LEU2 marker) to form plasmid pMCS-URA-TEF-hrGFP. The UAS1B₁₆-TEF-CAD expression cassette was gel extracted from plasmid pMCS-UAS1B₁₆-TEF-CAD and inserted into pMCS-URA-TEF-hrGFP with BstBI/AscI (replacing TEF-hrGFP) to form plasmid pMCS-URA-UAS1B₁₆-TEF-CAD.

Primers JB1143/1144 (SEQ ID NOs: 14/15) were used to amplify Y. lipolytica's native, mitochondrial-targeted aconitase gene (YALI0D09361g) from PO1f gDNA template. The aconitase open reading frame was inserted it into pMCS-URA-UAS1B₁₆-TEF-CAD in place of CAD with an AscI/PacI digest to form plasmid pMCS-URA-UAS1B₁₆-TEF-ACO. Similarly, primers JB1145/1144 (SEQ ID NOs: 16/15) amplified a truncated version of the aconitase gene (ACOnoMLS), removed of its mitochondrial localization signal (MLS) to prevent protein localization in the mitochondria. Insertion into pMCS-URA-UAS1B₁₆-TEF-CAD yielded pMCS-URA-UAS1B₁₆-TEF-ACOnoMLS. A rabbit bifunctional cytosolic iron-regulatory and aconitase protein (IRP1) with a S711D mutation that inhibits citrate to isocitrate conversion but not isocitrate to cis-aconitate conversion (Pitula, J. S. et al., 2004) was codon optimized for expression in yeast and synthesized by Life Technologies. Primers JB1168/1169 (SEQ ID NOs: 19/20) amplified IRP1 for AscI/PacI insertion into pMCS-URA-UAS1B₁₆-TEF-CAD to form pMCS-URA-UAS1B₁₆-TEF-IRP1.

Primers JB1140/1141 (SEQ ID NOs: 11/12) amplified Y. lipolytica's citrate synthase gene (YALI0E02684g) from PO1f gDNA template for insertion into pMCS-UAS1B₁₆-TEF-CAD with an AscI/PacI digest to form plasmid pMCS-UAS1B₁₆-TEF-CIT. Similarly, primers JB1142/1141 (SEQ ID NOs: 13/12) amplified a citrate synthase gene truncated of its MLS (CITnoMLS) to enable construction of pMCS-UAS1B₁₆-TEF-CITnoMLS.

Primers JB883/865 (SEQ ID NOs: 2/1) amplified an EXP1-Hph-Cyclt hygromycin resistance expression cassette from plasmid pKO (Blazeck, J. et al., 2013a) for Nad/BglII mediated insertion into plasmid pMCS-UAS1B₁₆-TEF-Cre (Blazeck, J. et al., 2013a) in place of the leucine marker to form plasmid pMCS-HYG-UAS1B₁₆-TEF-Cre.

Primers LQ311/312 (SEQ ID NOs: 25/26) amplified Y. lipolytica's pfk gene from PO1f gDNA template for insertion into pMCS-UAS1B₁₆-TEF with an AscI/PacI digest to form plasmid pMCS-UAS1B₁₆-TEF-PFK. Primers LQ317/318 (SEQ ID NOs: 27/28) and LQ319/320 (SEQ ID NOs: 29/30) were used to mutate K731 to A or R respectively which were inserted into pMCS-UAS1B₁₆-TEF with an AscI/PacI digest to form plasmids pMCS-UAS1B₁₆-TEF-PFKA and pMCS-UAS1B₁₆-TEF-PFKR respectively. Primers AH115/116 (SEQ ID NOs: 31/32) amplified an organic acid resistance transporter (YALI0E10483g) from PO1f gDNA template for insertion into pMCS-UAS1B₁₆-TEF with an AscI/PacI digest to form plasmid pMCS-UAS1B₁₆-TEF-MOAT.

Primers AH117/118 (SEQ ID NOs: 33/34) amplified Y. lipolytica's citrate transporter protein (YALI0F26323g) PO1f gDNA template to exclude intronic DNA. This was used as the template for amplification by primers AH118/119 (SEQ ID NOs: 34/35) for insertion into pMCS-UAS1B₁₆-TEF with an AscI/PacI digest to form plasmid pMCS-UAS1B₁₆-TEF-CTP1.

Strain Construction

All strains containing genomic modifications were confirmed through gDNA extraction and PCR confirmation. An AMPD chromosomal expression strain utilizing the uracil auxotrophic marker had previously been constructed, referred to as PO1f uracil AMPD (Blazeck, J. et al., 2014). A chromosomal, NotI-HF linearized pUC-52-UAS1B₁₆-TEF-CAD expression cassette was transformed into Y. lipolytica PO1f and PO1f uracil⁺ AMPD to form strains: PO1f leucine⁺ CAD and PO1f leucine⁺ uracil⁺ AMPD CAD.

The leucine and uracil markers were removed from PO1f leucine⁺ CAD and PO1f leucine⁺ uracil⁺ AMPD CAD by transforming each strain with plasmid pMCS-HYG-UAS1B₁₆-TEF-Cre and cultivation in YPD hygromycin media. Replica plating on YPD-hyg, YSC-leu, and YSC-ura plates enabled isolation of PO1f CAD and PO1f AMPD CAD strains that were leucine and uracil auxotrophs.

When relevant, episomal expression is denoted with an “Epi” moniker in the strain name. PO1f-based strains episomally expression the CAD gene were creating by transforming PO1f with pMCS-UAS1B₁₆-TEF-CAD or pMCS-URA-UAS1B₁₆-TEF-CAD singly, in tandem, or in combination with the requisite blank plasmid (pMCS-Cen1 or pMCS-URA-Cen1) to fully complement PO1f's auxotrophies. Additionally, PO1f uracil⁺ AMPD was transformed with pMCS-UAS1B₁₆-TEF-CAD to form PO1f leucine⁺ uracil⁺ AMPD CAD Epi. Similarly, multi-copy overexpressions of the CAD gene were enabled through transformation of the leucine/uracil auxotrophic PO1f CAD or PO1f AMPD CAD strains with episomal CAD expression vectors.

Plasmids pMCS-Cen1, pMCS-UAS1B₁₆-TEF-CIT, pMCS-UAS1B₁₆-TEF-CITnoMLS, pMCS-URA-Cen1, pMCS-URA-UAS1B₁₆-TEF-ACO, pMCS-URA-UAS1B₁₆-TEF-ACOnoMLS, and pMCS-URA-UAS1B₁₆-TEF-IRP1 were transformed singly or in pairs into the leucine/uracil auxotrophic PO1f AMPD CAD strain to analyze the effect of aconitase and citrate synthase cytosolic or mitochondrial expression.

Additional strains studied in this example are listed in Table 2 below. Table 3 lists overexpressed enzymes.

TABLE 2
Additional strains studied.

| Strains | itaconic acid |
|---|---|
| CAD epi, CIT epi | 103.8980833 |
| CAD epi, IRP1 epi | 70.8289 |
| CAD epi CITnoMLS epi | 135.8638833 |

TABLE 3
Enzymes for overexpression (or modification for increased activity).

| | | | SEQ |
|---|---|---|---|
| Enzyme Name | Organism | Gene ID | ID NO |
| cis-aconitic acid | A. terreus | AB326105 | 36 |
| decarboxylase | | | |
| AMP Deaminase | Y. lipolytica | YALI0E11495g | 37 |
| S711D iron-regulatory | O. cuniculus | Q01059 | 38 |
| protein mutant | | | |
| aconitase | Y. lipolytica | YALI0D09361g | 39 |
| aconitase no MLS | Y. lipolytica | YALI0D09361g | 40 |
| citrate synthase | Y. lipolytica | YALI0E02684g | 41 |
| citrate synthase no MLS | Y. lipolytica | YALI0E02684g | 42 |
| small acid resistance | Y. lipolytica | YALI0E10483g | 43 |
| transporter | | | |
| citrate transport protein | Y. lipolytica | YALI0F26323g | 44 |
| phosphofructokinase | Y. lipolytica | YALI0D16357g | 45 |
| K->A phosphofructokinase | Y. lipolytica | YALI0D16357g | 46 |
| mutant | | | |
| K->R phosphofructokinase | Y. lipolytica | YALI0D16357g | 47 |
| mutant | | | |

Itaconic Acid Production and Media Optimization

Cultivation for itaconic acid production always entailed the following: Yarrowia lipolytica strains were cultivated for two days at 30° C. with constant agitation in 2 mL cultures of the appropriate YSC media and then reinoculated to an OD₆₀₀=0.005 in 15 mL media in 250 mL flasks and shaken at 30° C. at 225 rpm.

Itaconic acid production as a function of media formulation was first investigated by cultivation in varying concentrations of glucose and nitrogen in YSC media. These media formulations contained 0.79 g/L CSM, 1.7 g/L Yeast Nitrogen Base w/o amino acid and w/o (NH₄)₂SO₄ (Becton, Dickinson, and Company), and the following concentrations of glucose and ammonium—20 g/L and 1.365 g/L ammonium (5 g/L ammonium sulfate), 20 g/L glucose and 0.055 g/L ammonium (0.2 g/L ammonium sulfate), and 80 g/L and 1.365 g/L ammonium (5 g/L ammonium sulfate). The effect of amino acid supplementation was investigated by cultivation in minimal media formulations utilized 20 g/L glucose, 6.7 g/L Yeast Nitrogen Base w/o amino acids (1.7 g/L YNB and 5 g/L ammonium sulfate (1.365 g/L ammonium)), and uracil supplementation at 0.02 g/L if necessary. Minimal media formulation was then further optimized by adjusting nitrogen availability. Strains were cultivated 20 g/L and 1.365 g/L ammonium (5 g/L ammonium sulfate), 20 g/L glucose and 0.273 g/L ammonium (1.00 g/L ammonium sulfate), and 20 g/L and 0.1365 g/L ammonium (0.50 g/L ammonium sulfate) and analyzed for itaconic acid production.

Time Course of Itaconic Acid Production

Strains PO1f leucine⁺ CAD and PO1f leucine⁺ uracil⁺ AMPD CAD were cultivated in minimal media formulations utilizing 20 g/L and 1.365 g/L ammonium (5 g/L ammonium sulfate) with uracil supplementation if necessary for seven days and analyzed for itaconic acid production, citric acid production, and OD₆₀₀ after two, three, four, six, and seven days.

Citric Acid and Itaconic Acid Quantification

A 1-2 mL culture sample was pelleted down for 5 minutes at 3000×g, and the supernatant was filtered using a 0.2 mm syringe filter (Corning Incorporated). Filtered supernatant was analyzed with a HPLC Ultimate 3000 (Dionex) and a Zorbax SB-Aq column (Agilent Technologies). A 2.0 μL injection volume was used in a mobile phase composed of a 99.5:0.5 ratio of 25 mM potassium phosphate buffer (pH=2.0) to acetonitrile with a flow rate of 1.25 mL/min. The column temperature was maintained at 30° C. and UV-Vis absorption was measured at 210 nm. Citric acid and itaconic acid standards (Sigma-Aldrich) were used to detect and quantify organic acid production.

Prediction of Intracellular Localization

Probability of mitochondrial protein localization was predicted using the MITOPROP II v1.101 program (Claros, M. G. et al., 1996). In all cases, the entire protein's amino acid sequence was inputted.

Bioreactor Fermentations

Typically, bioreactor fermentations were run in minimal media containing 80 g/L glucose and 6.7 g/L Yeast Nitrogen Base w/o amino acids as batch processes. All fermentations were inoculated to an initial OD₆₀₀=0.1 in 1.5 L of media. Dissolved oxygen was maintained at 50% of maximum by varying rotor speed between 250 rpm and 800 rpm with a constant air input flow rate of 2.5 v v⁻¹ min⁻¹ (3.75 L min⁻¹). PH was maintained at 3.5 or above with 2.5 M NaOH, and temperature was maintained at 28° C. 10-15 mL samples were taken every twenty-four hours, and fermentations lasted 7 days. The inventors ran several fermentations with suboptimal conditions before settling on the above parameters.

pH Tolerance Adaptive Evolution

PO1f, S1, S2 CAD, AMPD CAD, and AMPD CAD CADₑₚᵢ ACONOMLSₑₚᵢ strains were subjected to serial re-culturing in YSC or YSC-LEU,-URA media, depending on the presence of episomal plasmids. With each subsequent transfer, the initial pH of the media was decreased by 0.1 points using HCl, starting with a initial pH of 5.0 and terminating with an initial pH of 2.8. Cells were grown in 20 mL of appropriate media in 250 mL flasks at 30° C. at 225 rpm. Cells were transferred during late exponential phase into fresh media with a 1000-fold dilution. Once the adaption was completed, the native and evolved strains were tested for improved growth in low-pH conditions. For this test, the native strains and isolates from various stages of the adaption were initially inoculated into 3 mL of YSC or YSC-LEU,-URA media and cultured for 3 days at 30° C. in triplicate. The strains were then inoculated at an OD₆₀₀ of 0.01 into 2 mL of YSC or YSC-LEU,-URA adjusted to an initial pH of 4.0, 3.5, 3.0, or 2.5 as well as an unadjusted control. After 24 hours, OD₆₀₀ measurements were periodically taken until 63 hours of fermentation.

Results and Discussion

Episomal expression of the CAD gene in Y. lipolytica

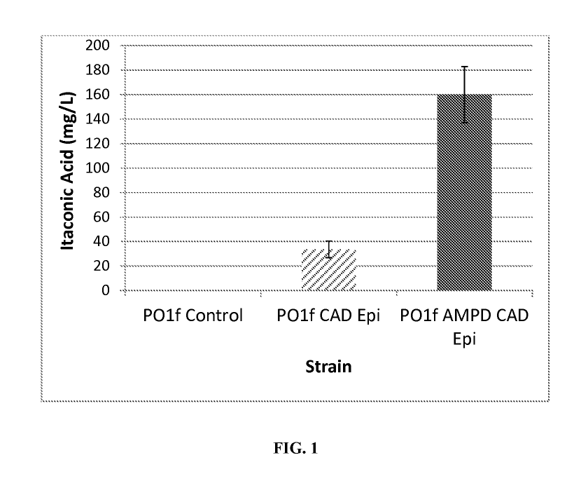

Recent characterization of the cis-aconitic acid decarboxylase gene (CAD) enables its utilization for itaconic acid production in microbial hosts. The inventors inserted the CAD gene into a high-strength UAS1B₁₆-TEF expression cassette on an episomal plasmid to allow for expression in Y. lipolytica, and 33 mg/L itaconic acid titer was observed (FIG. 1). This represents the first time that itaconic acid has been produced by Y. lipolytica and illustrates that CAD expression can enable itaconic acid production in Y. lipolytica. The inventors note that the CAD gene had not been codon optimized from its original codon usage in Aspergillus terreus. In fact, use of a CAD optimized for S. cerevisiae expression resulted in no itaconic acid production in Y. lipolytica. This demonstrates the previously described importance of codon usage for heterologous protein expression in Y. lipolytica (Blazeck, J. et al., 2011).

The inventors attempted to increase itaconic acid production by expressing CAD (again episomally) in a Y. lipolytica strain with the AMP Deaminase (AMPD) enzyme constitutively overexpressed in a UAS1B₁₆-TEF-driven chromosomal expression cassette. Constitutive expression of AMPD inhibits the citric acid cycle at the isocitric acid intermediate, increasing cis-aconitic acid substrate levels (Beopoulos, A. et al., 2009b). A nearly fivefold increase in itaconic acid was observed in this AMPD overexpression background strain, to 159 mg/L (FIG. 1). Thus, AMPD overexpression increased itaconic acid production through inhibition of the TCA cycle to increase organic acid substrate levels.

Optimizing C:N Ratio for Itaconic Acid Production

As described above, Y. lipolytica's central carbon metabolism is pliable to manipulation by AMPD overexpression. It have been previously demonstrated that Y. lipolytica's lipid accumulation potential can be manipulated by controlling carbon (glucose) and nitrogen (ammonium) availability in media formulations (C:N ratio) (Blazeck, J. et al., 2013b). High C:N ratios promotes citric acid accumulation (a metabolic precursor for cis-aconitic acid CAD substrate) by stimulating a nitrogen starvation response that inhibits the citric acid cycle through AMPD-mediated activity (Beopoulos, A. et al., 2009a; Beopoulos, A. et al., 2009b).

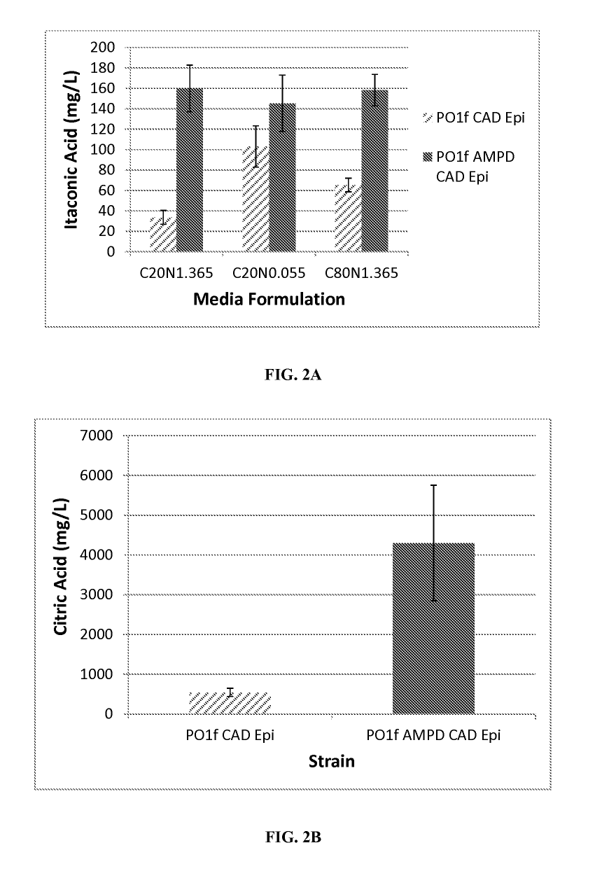

Thus, the inventors attempted to increase citric acid and itaconic acid production by cultivating Y. lipolytica PO1f and PO1f AMPD strains, harboring episomal CAD expression cassettes, in media formulations with increased C:N ratio (FIG. 2A). Two formulations containing 20 g/L glucose and 0.055 g/L ammonium (C₂₀N_{0.055}) or 80 g/L glucose and 1.365 g/L ammonium (C₈₀N_{1.365}), were compared to the initial formulation—20 g/L glucose and 1.365 g/L ammonium (C₂₀N_{1.365}). All three formulations also contained yeast nitrogen base and CSM-leucine amino acid supplementation. Increasing C:N ratio improved itaconic acid production to more than 100 mg/L in the unmodified Y. lipolytica background, but had little benefit when the AMPD enzyme was coexpressed (FIG. 2A). This confirmed that AMPD overexpression and nitrogen starvation have similar mechanisms to inhibit the TCA cycle to increase organic acid levels. These two methods did not cooperatively effect an increase in itaconic acid production in the PO1f AMPD background when employed simultaneously, instead resulting in drastic citric acid buildup to more than 4 g/L (FIGS. 2A-2B). Only the C₂₀N_{0.055} media formulation resulted in citric acid accumulation, demonstrating that severe nitrogen limitation is necessary for complete TCA cycle inhibition (FIG. 2B). The two tested media formulations, C₈₀N_{1.365} and C₂₀N_{0.055}, represent upper and lower boundaries of carbon and nitrogen levels. Thus, there is a possibility that more fine-tune media formulation manipulation could enhance itaconic acid production in the PO1f AMPD background.

Integration of CAD to increase itaconic acid production

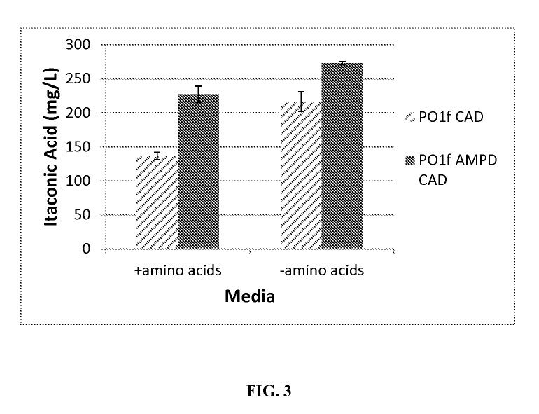

Increased protein activity using chromosomal expression compared to episomal expression in Y. lipolytica for a hrGFP reporter gene has previously been observed (Blazeck, J. et al., 2011). Therefore, the inventors integrated the CAD gene into the PO1f and PO1f AMPD overexpression backgrounds and assayed for itaconic acid production (FIG. 3). A pronounced increase in itaconic acid production was observed, suggesting that CAD expression is a limiting factor in the systems of the invention.

The inventors assayed the two chromosomal CAD expression strains for itaconic acid when cultivated in minimal media (C₂₀N_{1.365}—amino acids). The PO1f chromosomal CAD expression strain required additional supplementation with 20 mg/L due to a uracil auxotrophy that had been alleviated in the PO1f AMPD overexpression background by insertion of the AMPD expression cassette (Blazeck, J. et al., 2014). Another pronounced increase in itaconic acid production was observed, culminating in 272 mg/L produced by the AMPD overexpression background (FIG. 3). Thus, eliminating amino acid supplementation increased itaconic acid production in Y. lipolytica independent of strain background.

Optimizing Cultivation Duration

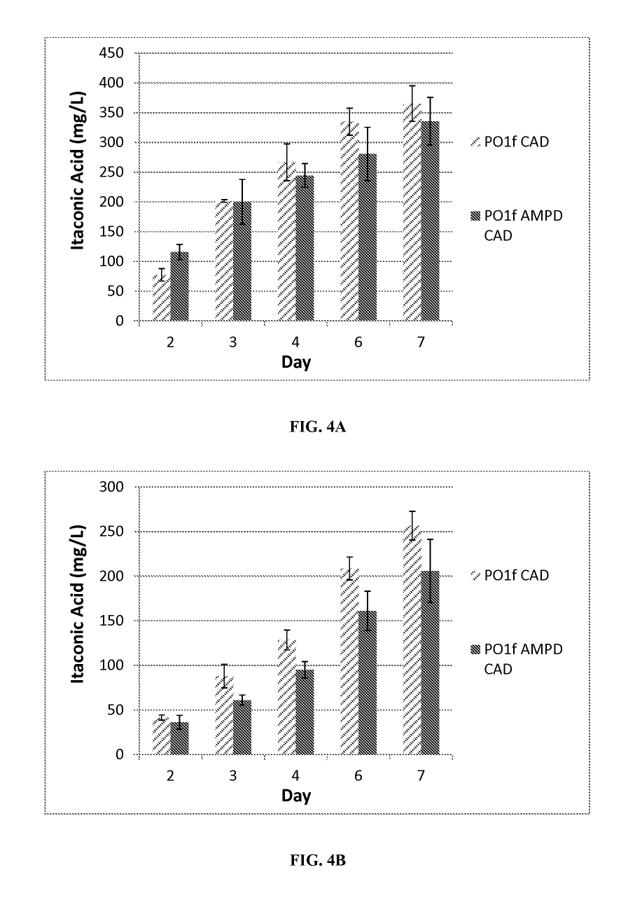

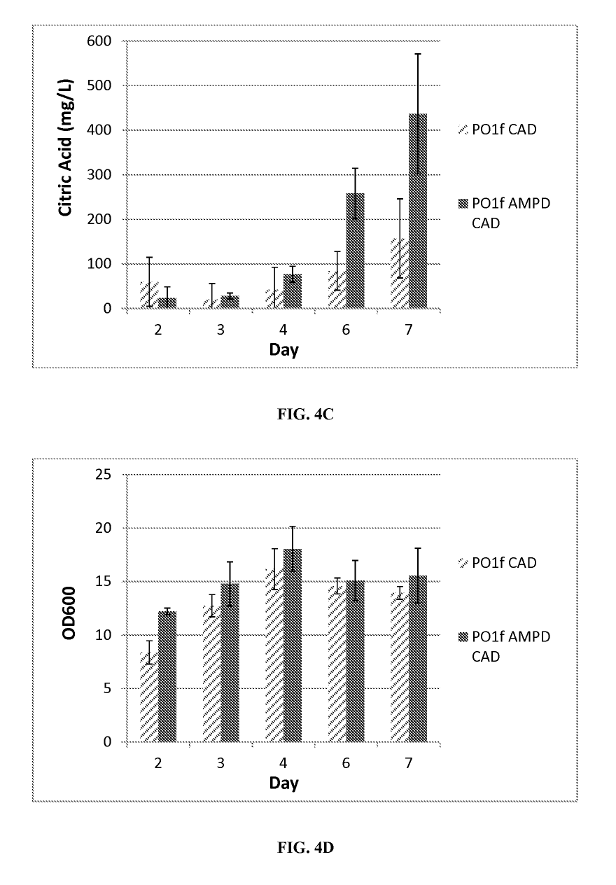

The inventors analyzed itaconic acid and citric acid production of the PO1f CAD and PO1f AMPD CAD chromosomal expression strains in C₂₀N_{1.365} minimal media (no amino acid supplementation) for seven days (FIG. 4A). A steady increase in itaconic acid was observed for both strains throughout the cultivation period to the highest titer yet observed (FIG. 4A), but no citric acid accumulation was observed. Similar cultivation in C₂₀N_{0.055} minimal media reduced itaconic acid production (FIG. 4B), but did increase citric acid production to 437 mg/L in the PO1f AMPD CAD strain (FIG. 4C). This substantial decrease in citric acid accumulation compared to the prior results during cultivation in standard C₂₀N_{0.055} media (with amino acids) revealed that amino acid supplementation promotes extracellular citric acid accumulation, but not intracellular flux towards TCA cycle derived metabolites, as seen here with itaconic acid and as previously seen with fatty acid accumulation (Blazeck, J. et al., 2014). Growth curves reveal that both strains were fully grown after 3-4 days (FIG. 4D), demonstrating that itaconic acid production occurs independent of cell growth phase.

Fine-Tuning Media Formulation to Increase Itaconic Acid Production

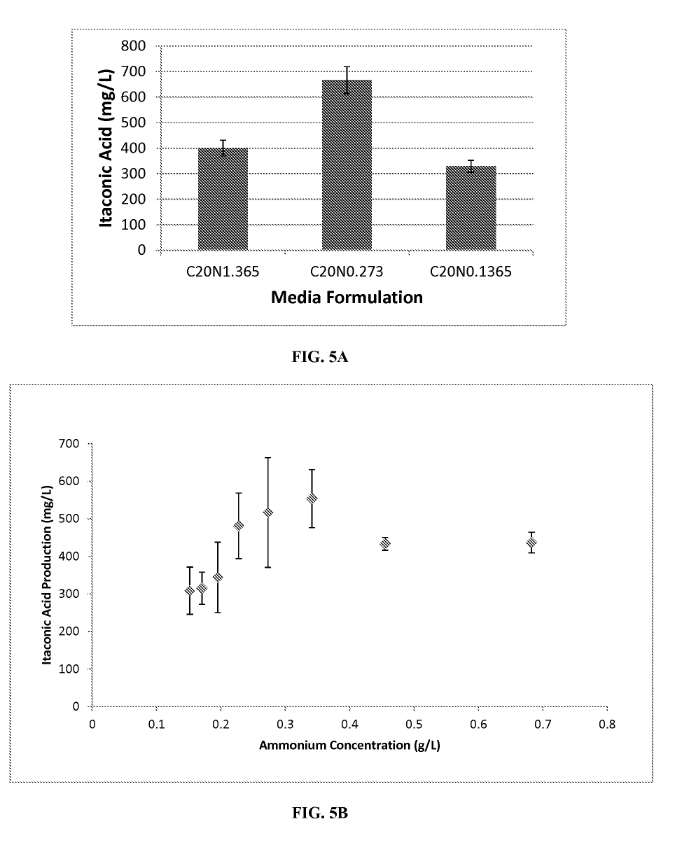

The inventors attempted to further modify media formulation utilizing drastic adjustments in carbon and nitrogen availability and failed to increase itaconic acid production in the PO1f AMPD background. In some studies media formulation enhanced by reducing nitrogen content less severely. The PO1f AMPD CAD strain was cultivated for seven days in three minimal media formulations, C₂₀N_{1.365}, C₂₀N_{0.273}, and C₂₀N_{0.1365}. Reducing nitrogen availability by 80% (i.e., a C:N molar ratio of ˜44 at 20 g/L of glucose) using the C₂₀N_{0.273} media formulation drastically increased itaconic acid production to 667 mg/L (FIG. 5A). A 90% reduction in nitrogen content (i.e., a C:N molar ratio of ˜88 at 20 g/L of glucose) abrogated this effect (FIG. 5A). In this regard, nitrogen reduction and AMPD overexpression can exhibit cooperative effects towards increasing itaconic acid production, provided the nitrogen reduction is subtle enough. Further testing of intermediate reductions in nitrogen content were performed to fully optimize itaconic acid production in this PO1f AMPD CAD strain (FIG. 5B); however, the test identified the C₂₀N_{0.273} media formulation as optimal for itaconic acid production

Bioreactor Fermenations

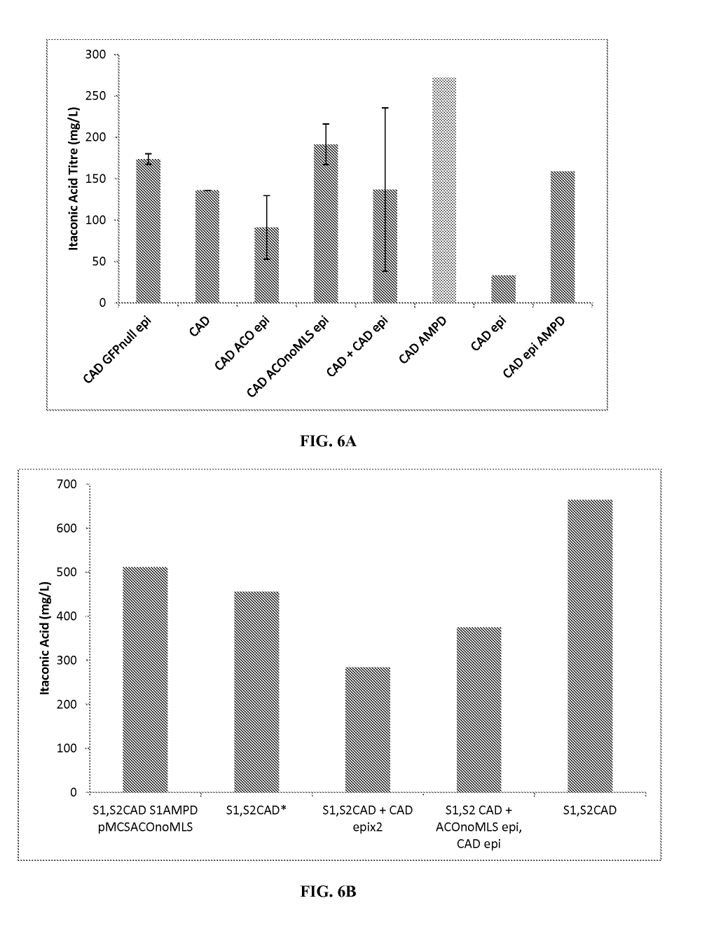

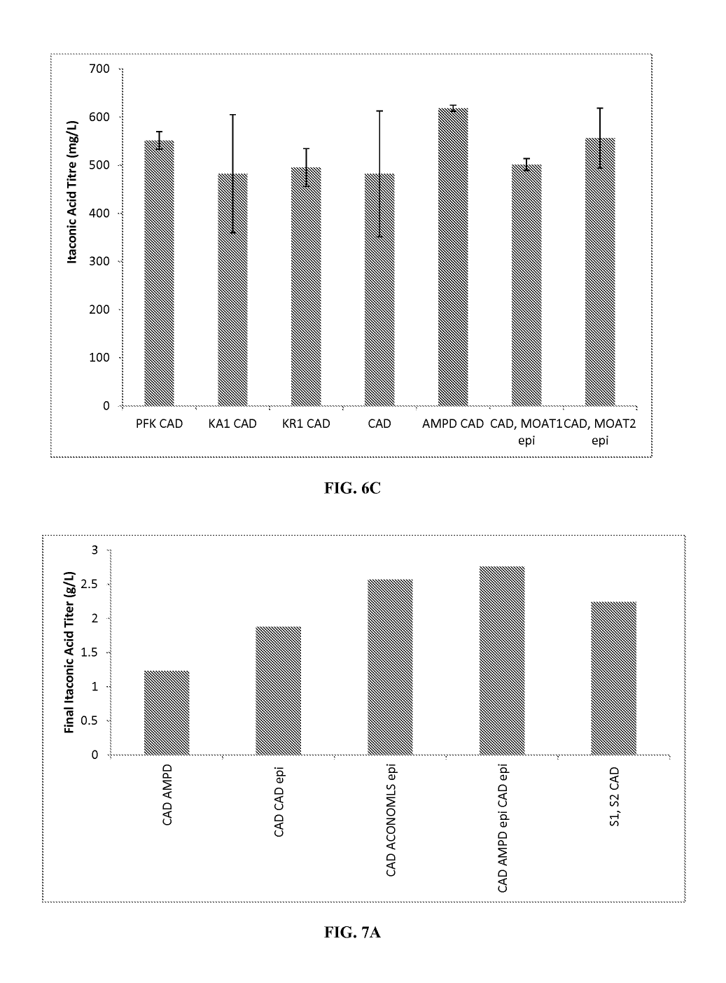

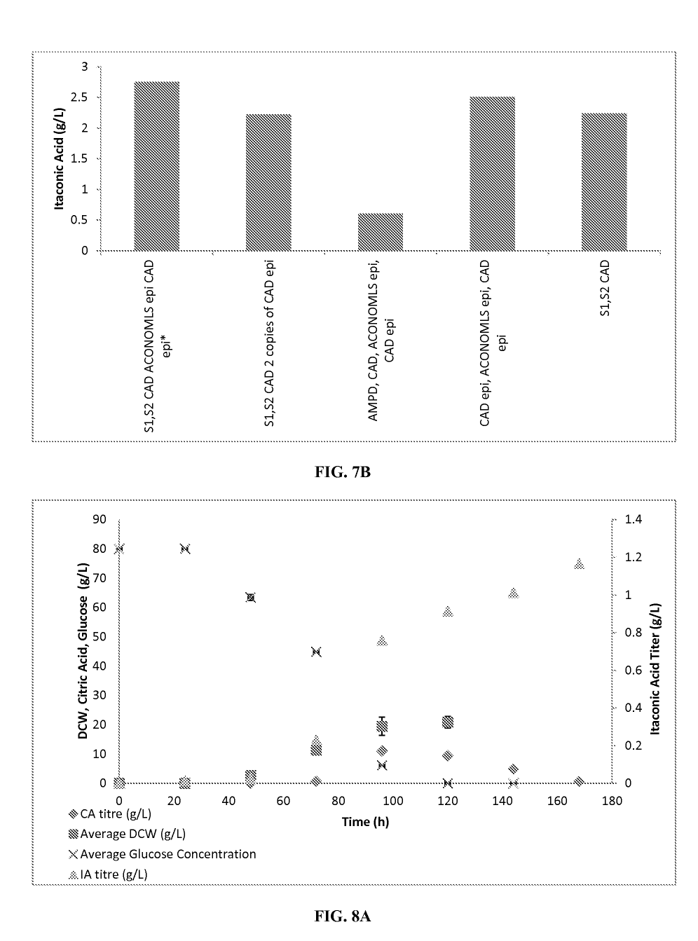

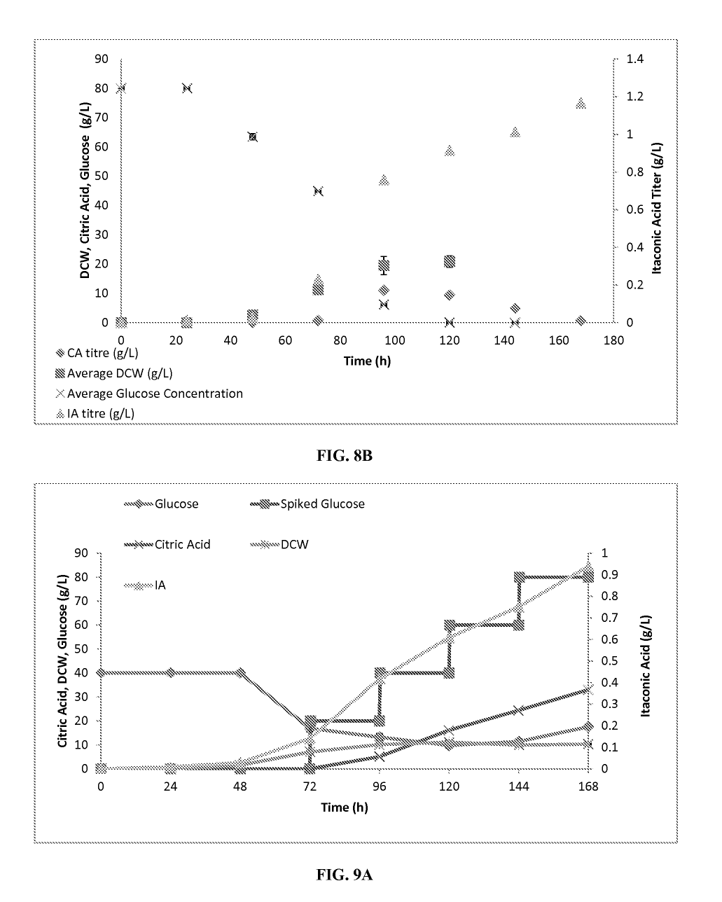

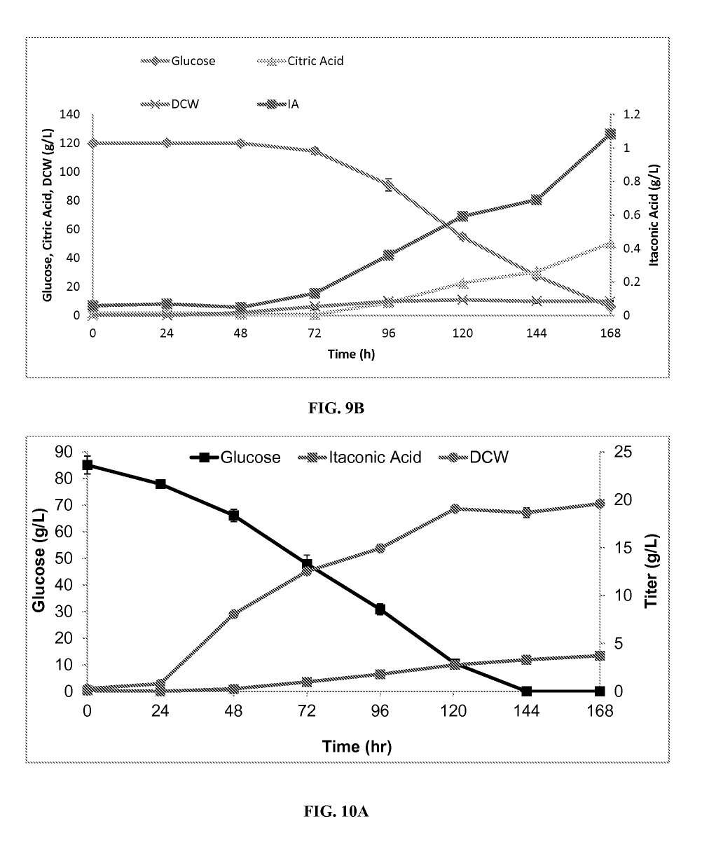

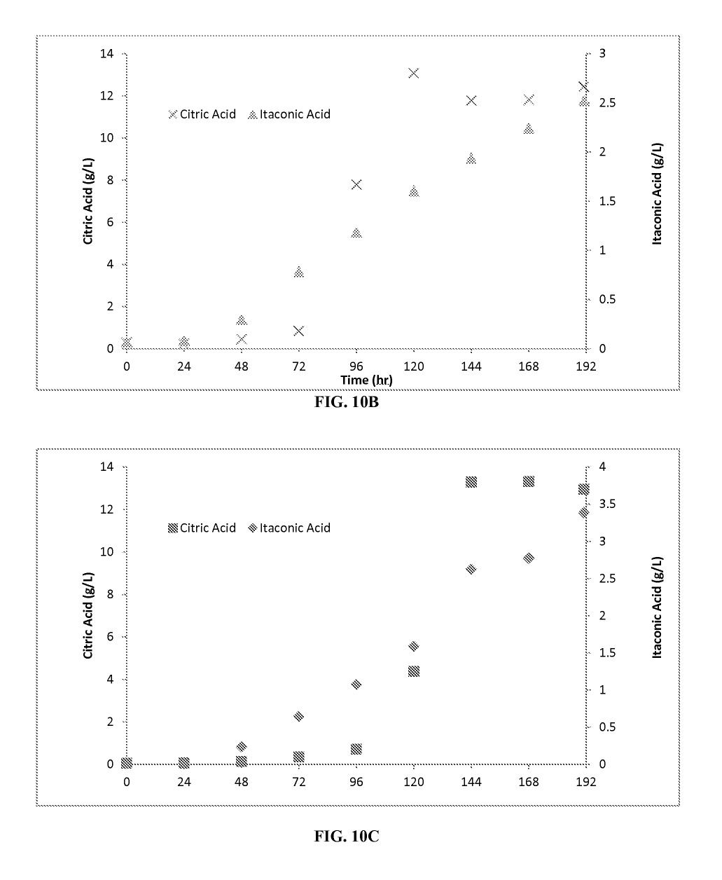

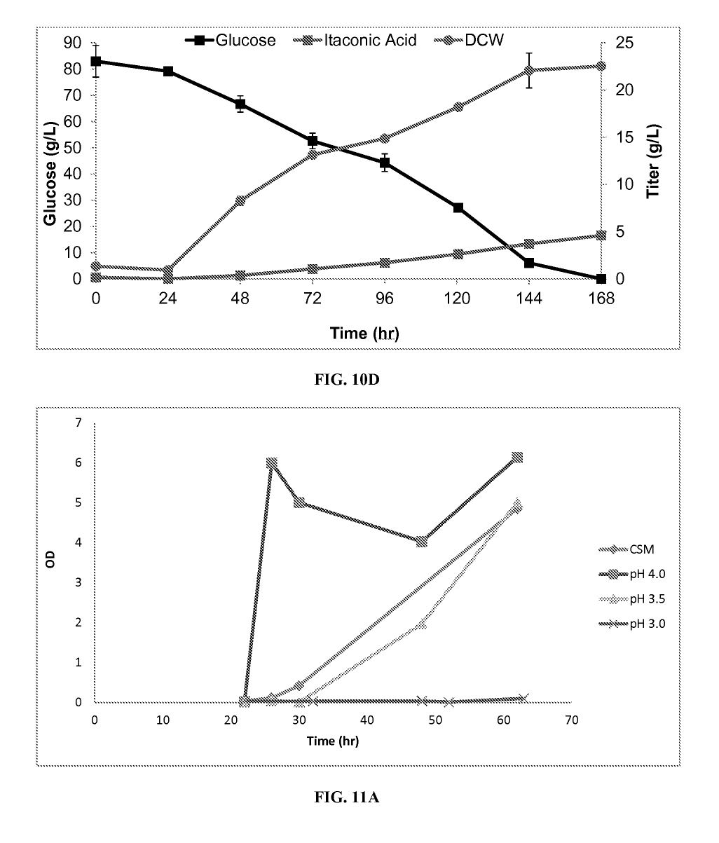

Various strains containing combinations of AMPD, CAD, and aconitase, mitochrondrial organic acid transporters (MOATs), and phosphofructokinases, and ACOnoMLS overexpressions were tested for itaconic acid production in flask-scale fermentations to determine optimal strains for bioreactor fermenations (FIGS. 6A-6C). Several of these strains were ultimate evaluated in bioreactor fermentations to determine their ability to produce itaconic acid (FIGS. 7A-7B). Surprisingly, the best performing strain in flask scale fermentations, PO1f AMPD CAD, performed relatively poorly in bioreactor fermentations, producing only 1.2 g/L itaconic acid (FIGS. 8A-8B). Variations of fermentations failed to improve the itaconic acid titer, despite the production of 30 g/L and 50 g/L of citric acid depending on whether the bioreactor was spiked (FIG. 9A) or not (FIG. 9B). The best itaconic acid producing strain in bioreactor fermentations was the 51, S2 CAD ACONOMLS epi, CAD epi strain, with an itaconic acid titer of 4.6 g/L (FIG. 10D). This titer represents a 140-fold improvement over the initial strain and conditions, which can be further improved with additional optimization.

pH Growth Adaption

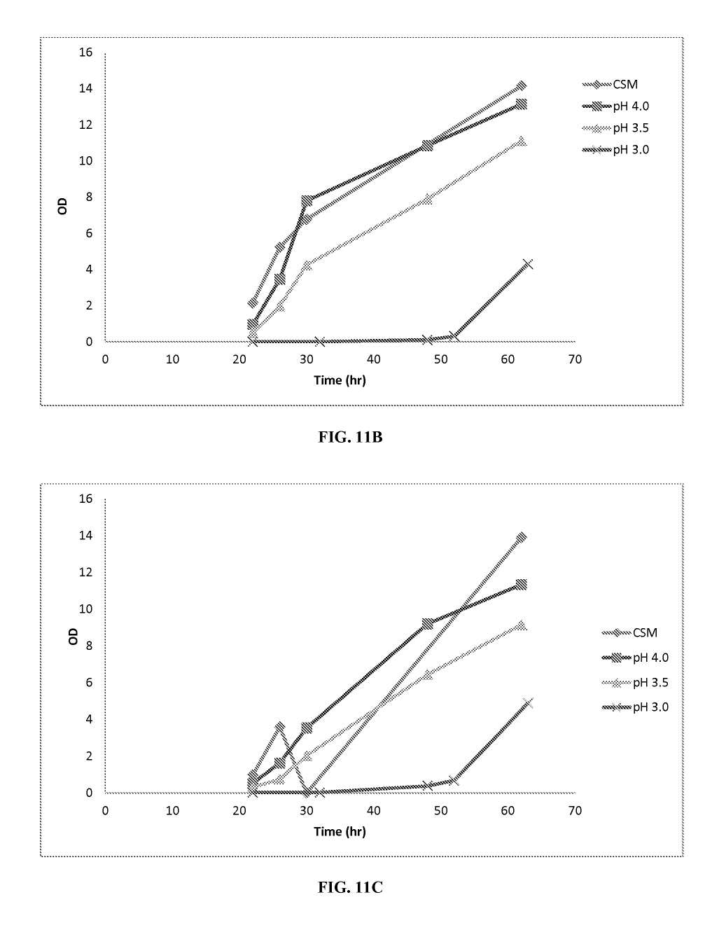

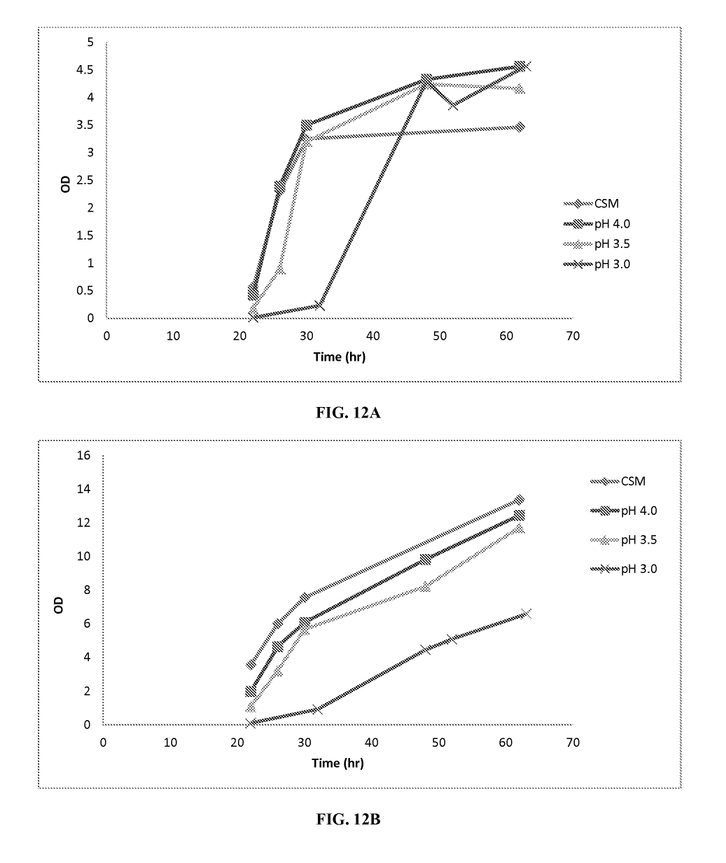

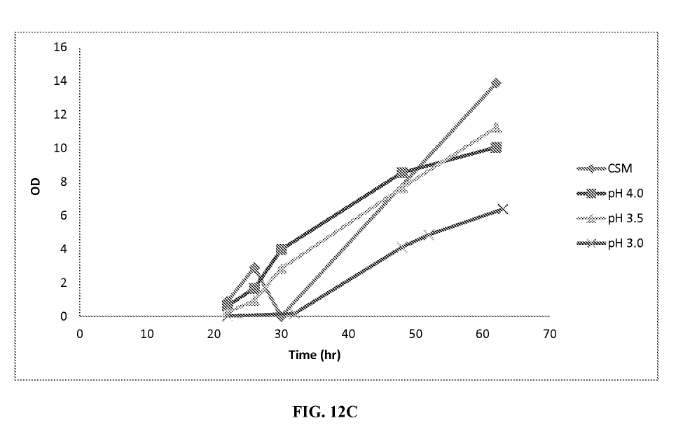

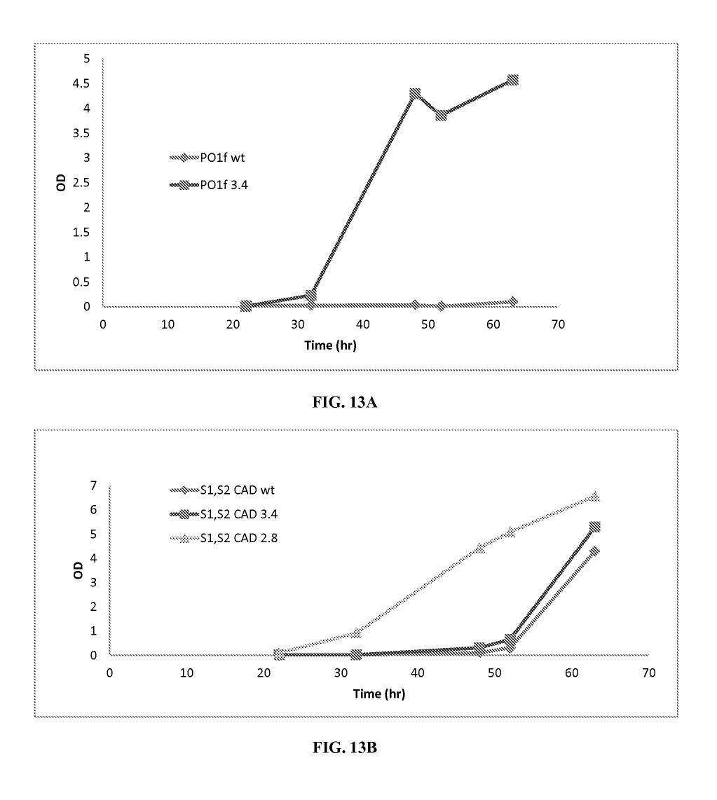

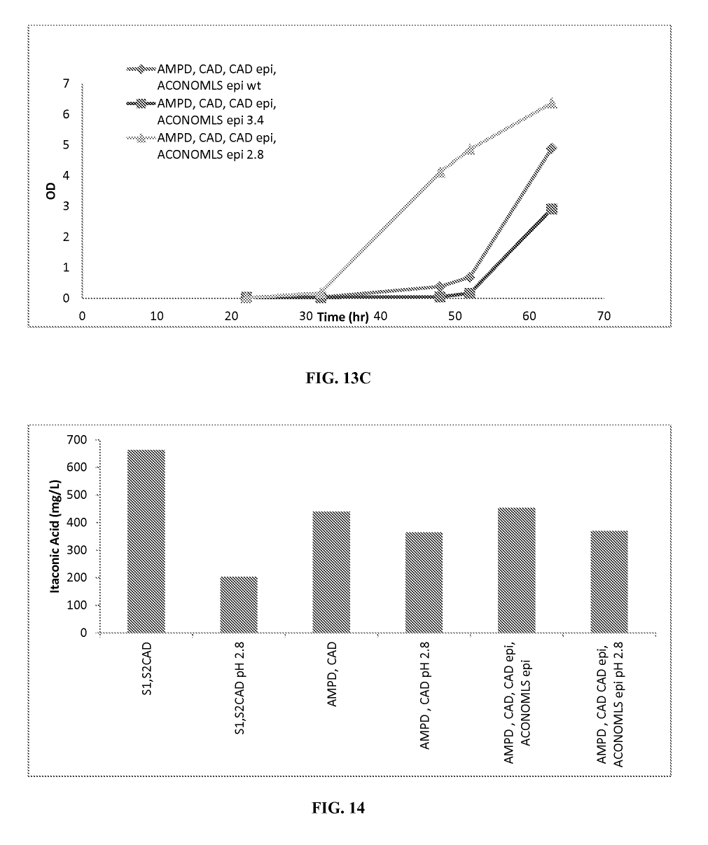

Evaluating the growth curves of both the native strains (FIGS. 11A-11C), as well as the strains evolved for growth in low-pH conditions (FIGS. 12A-12C) indicated that evolution had little effect except when the initial pH was 3.0. For the cultures grown in media adjusted to an initial pH of 3.0, there was an observed lag phase of at least 24 hours and the native PO1f strain was unable to grow entirely. Observing the growth curves of the natives strains compared to the mutants isolated from the low pH adaption experiment in these conditions demonstrates that only the evolved mutants, particularly those isolated from more extreme conditions (initial pH: 2.8) were able to demonstrate a shortened lag phase (FIGS. 13A-13C). However, with the exception of the PO1f strain, the other native strains were able to eventually catch up with the growth of the adapted strains. When tested for the production of itaconic acid, adapted strains generally saw mild to severe reductions in the production of itaconic acid (FIG. 14).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Barth, G., Gaillardin, C., 1996. Yarrowia lipolytica. In: Wolf, K., (Ed.), Nonconventional Yeasts in Biotechnology: A Handbook. vol. 1. Springer, pp. 313-388.
Beopoulos, A., Cescut, J., Haddouche, R., Uribelarrea, J. L., Molina-Jouve, C., Nicaud, J. M., 2009a. Yarrowia lipolytica as a model for bio-oil production. Prog. Lipid Res. 48, 375-387.
Beopoulos, A., Chardot, T., Nicaud, J. M., 2009b. Yarrowia lipolytica: A model and a tool to understand the mechanisms implicated in lipid accumulation. Biochimie. 91, 692-696.
Beopoulos, A., Mrozova, Z., Thevenieau, F., Le Dall, M. T., Hapala, I., Papanikolaou, S., Chardot, T., Nicaud, J. M., 2008. Control of Lipid Accumulation in the Yeast Yarrowia lipolytica. Applied and Environmental Microbiology. 74, 7779-7789.
Blazeck, J., Garg, R., Reed, B., Alper, H., 2012. Controlling promoter strength and regulation in Saccharomyces cerevisiae using synthetic hybrid promoters. Biotechnology and Bioengineering. 109, 2884-2995.
Blazeck, J., Hill, A., Liu, L., Knight, R., Miller, J., Pan, A., Otoupal, P., Alper, H. S., 2014. Harnessing Yarrowia lipolytica lipogenesis to create a platform for lipid and biofuel production. Nature Communications. 5.
Blazeck, J., Liu, L., Knight, R., Alper, H., 2013a. Heterologous production of pentane in the oleaginous yeast Yarrowia lipolytica. Journal of Biotechnology. 165, 184-194.
Blazeck, J., Liu, L., Knight, R., Alper, H. S., 2013b. Heterologous production of pentane in the oleaginous yeast Yarrowia lipolytica. Journal of Biotechnology. 165, 184-194.
Blazeck, J., Liu, L., Redden, H., Alper, H., 2011 Tuning Gene Expression in Yarrowia lipolytica by a Hybrid Promoter Approach. Applied and Environmental Microbiology. 77, 7905-7914
Blazeck, J., Reed, B., Garg, R., Gerstner, R., Pan, A., Agarwala, V., Alper, H., 2013c. Generalizing a hybrid synthetic promoter approach in Yarrowia lipolytica. Applied Microbiology and Biotechnology. 97, 3037-3052.
Bonnarme, P., Gillet, B., Sepulchre, A. M., Role, C., Beloeil, J. C., Ducrocq, C., 1995. Itaconate Biosynthesis in Aspergillus terreus. Journal of Bacteriology. 177, 3573-3578.
Claros, M. G., Vincens, P., 1996. Computational method to predict mitochondrially imported proteins and their targeting sequences. European Journal of Biochemistry. 241, 779-786.
Fickers, P., Le Dall, M. T., Gaillardin, C., Thonart, P., Nicaud, J. M., 2003. New disruption cassettes for rapid gene disruption and marker rescue in the yeast Yarrowia lipolytica. J. Microbiol. Methods. 55, 727-737.
Finogenova, T. V., Morgunov, I. G., Kamzolova, S. V., Chernyayskaya, O. G., 2005. Organic acid production by the yeast Yarrowia lipolytica: A review of prospects. Applied Biochemistry and Microbiology. 41, 418-425.
Gon, #xe7, alves, F. A. G., Colen, G., Takahashi, J. A., 2014. Yarrowia lipolytica and Its Multiple Applications in the Biotechnological Industry. The Scientific World Journal. 2014, 14.
Guldener, U., Heck, S., Fiedler, T., Beinhauer, J., Hegemann, J. H., 1996. A new efficient gene disruption cassette for repeated use in budding yeast. Nucleic Acids Research. 24, 2519-2524.
Hevekerl, A., Kuenz, A., Vorlop, K.-D., 2014. Filamentous fungi in microtiter plates—an easy way to optimize itaconic acid production with Aspergillus terreus. Applied Microbiology and Biotechnology. 1-7.
Itaconix, L., The development of integrated production of polyitaconic acid from Northeast hardwood biomass. NIFA, New Hampshire, 2009.
Juretzek, T., Le Dall, M. T., Mauersberger, S., Gaillardin, C., Barth, G., Nicaud, J. M., 2001. Vectors for gene expression and amplification in the yeast Yarrowia lipolytica. Yeast. 18, 97-113.
Kamzolova, S. V., Shishkanova, N. V., Morgunov, I. G., Finogenova, T. V., 2003. Oxygen requirements for growth and citric acid production of Yarrowia lipolytica. Fems Yeast Research. 3, 217-222.

Kanamasa, S., Dwiarti, L., Okabe, M., Park, E. Y., 2008. Cloning and functional characterization of the cis-aconitic acid decarboxylase (CAD) gene from Aspergillus terreus. Applied Microbiology and Biotechnology. 80, 223-229.
Kautola, H., Rymowicz, W., Linko, Y. Y., Linko, P., 1991. Itaconic acid production by immobilized Aspergillus terreus with varied metal additions. Applied Microbiology and Biotechnology. 35, 154-158.
Kinoshita, K., 1932. Über die Production von Itaconsäure and Mannit durch einem neuen Schimmelpilz Aspergillus itaconicus. Acta Phytochim. 5, 271-287.
Li, A., Pfelzer, N., Zuijderwijk, R., Punt, P., 2012. Enhanced itaconic acid production in Aspergillus niger using genetic modification and medium optimization. Bmc Biotechnology. 12.
Madzak, C., Gaillardin, C., Beckerich, J. M., 2004. Heterologous protein expression and secretion in the non-conventional yeast Yarrowia lipolytica: a review. Journal of Biotechnology. 109, 63-81.
Madzak, C., Treton, B., Blanchin-Roland, S., 2000. Strong hybrid promoters and integrative expression/secretion vectors for quasi-constitutive expression of heterologous proteins in the yeast Yarrowia lipolytica. J. Mol. Microbiol. Biotechnol. 2, 207-216.
Morgunov, I., Kamzolova, S., Lunina, J., 2013. The citric acid production from raw glycerol by Yarrowia lipolytica yeast and its regulation. Applied Microbiology and Biotechnology. 97, 7387-7397.
Nuss, P., Gardner, K. H., 2013. Attributional life cycle assessment (ALCA) of polyitaconic acid production from northeast US softwood biomass. International Journal of Life Cycle Assessment. 18, 603-612.
Okabe, M., Lies, D., Kanamasa, S., Park, E. Y., 2009. Biotechnological production of itaconic acid and its biosynthesis in Aspergillus terreus. Applied Microbiology and Biotechnology. 84, 597-606.
Papanikolaou, S., Chatzifragkou, A., Fakas, S., Galiotou-Panayotou, M., Komaitis, M., Nicaud, J. M., Aggelis, G., 2009. Biosynthesis of lipids and organic acids by Yarrowia lipolytica strains cultivated on glucose. European Journal of Lipid Science and Technology. 111, 1221-1232.
Papanikolaou, S., Muniglia, L., Chevalot, I., Aggelis, G., Marc, I., 2002. Yarrowia lipolytica as a potential producer of citric acid from raw glycerol. Journal of Applied Microbiology. 92, 737-744.
Park, Y. S., Itida, M., Ohta, N., Okabe, M., 1994. Itaconic acid production using an air-lift bioreactor in repeated batch culture of Aspergillus terreus. Journal of Fermentation and Bioengineering. 77, 329-331.
Pitula, J. S., Deck, K. M., Clarke, S. L., Anderson, S. A., Vasanthakumar, A., Eisenstein, R. S., 2004. Selective inhibition of the citrate-to-isocitrate reaction of cytosolic aconitase by phosphomimetic mutation of serine-711. Proceedings of the National Academy of Sciences of the United States of America. 101, 10907-10912.
Rywinska, A., Musial, I., Rymowicz, W., Zarowska, B., Boruczkowski, T., 2012. Effect of agitation and aeration on the citric acid production by Yarrowia lipolytica grown on glycerol. Preparative Biochemistry & Biotechnology. 42, 279-291.
Rywinska, A., Rymowicz, W., 2010. High-yield production of citric acid by Yarrowia lipolytica on glycerol in repeated-batch bioreactors. Journal of Industrial Microbiology & Biotechnology. 37, 431-435.
Rywinska, A., Rymowicz, W., Zarowska, B., Wojtatowicz, M., 2009. Biosynthesis of Citric Acid from Glycerol by Acetate Mutants of Yarrowia lipolytica in Fed-Batch Fermentation. Food Technology and Biotechnology. 47, 1-6.
Shao, Z., Zhao, H., Zhao, H., 2009. DNA assembler, an in vivo genetic method for rapid construction of biochemical pathways. Nucleic Acids Research. 37, e16.
Tate, B. E., Itaconic acid and derivatives. In: Grayson, M., Eckroth, E., Eds.), Kirk-Othmer Encyclopedia of Chemical Technology, 1981, pp. 865-873.
Tevz, G., Bencina, M., Legisa, M., 2010. Enhancing itaconic acid production by Aspergillus terreus. Applied Microbiology and Biotechnology. 87, 1657-1664.
Tsai, Y. C., Huang, M. C., Lin, S. F., Su, Y. C., Method for the production of itaconic acid using aspergillus terreus solid state fermentation. Vol. U.S. Pat. No. 6,171,831 B1. National Science Council, United States, 2000.
van der Straat, L., Vernooij, M., Lammers, M., van den Berg, W., Schonewille, T., Cordewener, J., van der Meer, I., Koops, A., de Graaff, L., 2014. Expression of the Aspergillus terreus itaconic acid biosynthesis cluster in Aspergillus niger. Microbial Cell Factories. 13, 11.
Werpy, T., Petersen, G., Top Value Added Chemicals from Biomass: Volume I—Results of Screening for Potential Candidates from Sugars and Synthesis Gas. Vol. 1. U.S Department of Energy, 2004.
Yahiro, K., Takahama, T., Park, Y. S., Okabe, M., 1995. Breeding of Aspergillus terreus Mutant TN-484 for Itaconic Acid Production with High Yield. Journal of Fermentation and Bioengineering. 79, 506-508.

What is claimed is:

1. A transgenic oleaginous fungus, the fungus comprising at least a first transgenic nucleic acid molecule encoding a cis-aconitic acid decarboxylase (CAD) enzyme operably linked to a promoter functional in the fungus and at least a second genetic modification that increases expression or activity of a gene product selected from the group consisting of AMP deaminase (AMPD), iron-regulatory protein, aconitase, citrate synthase, small acid resistance transporter, citrate transport protein and phosphofructokinase.

2. The fungus of claim 1, wherein the oleaginous fungus is Yarrowia lipolytica.

3. The fungus of claim 1, wherein the fungus comprises a genome integrated nucleic acid molecule encoding a CAD enzyme operably linked to a promoter functional in the fungus.

4. The fungus of claim 1, wherein the fungus comprises an episomal nucleic acid molecule encoding a CAD enzyme operably linked to a promoter functional in the fungus.

5. The fungus of claim 3, wherein the fungus comprises a genome integrated and an episomal nucleic acid molecule each encoding a CAD enzyme operably linked to a promoter functional in the fungus.

6. The fungus of claim 1, wherein the second genetic modification comprises introduction of an expressible transgene encoding a gene product selected from the group consisting of AMPD, iron-regulatory protein, aconitase, citrate synthase, small acid resistance transporter, citrate transport protein and phosphofructokinase.

7. The fungus of claim 1, wherein the second genetic modification comprises mutation or replacement of a promoter linked to an AMPD, iron-regulatory protein, aconitase, citrate synthase, small acid resistance transporter, citrate transport protein or phosphofructokinase gene in the fungus.

8. The fungus of claim 1, wherein the second genetic modification comprises mutation of the coding sequence for an AMPD, iron-regulatory protein, aconitase, citrate synthase, small acid resistance transporter, citrate transport protein or phosphofructokinase gene that increased activity of the gene product.

9. The fungus of claim 1, further comprising at least third, fourth, fifth or sixth genetic modification that increases expression or activity of a gene product selected from the group consisting of AMPD, iron-regulatory protein, aconitase, citrate synthase, small acid resistance transporter, citrate transport protein and phosphofructokinase.

10. The fungus of claim 1, further comprising a transgene encoding a selectable or screenable marker.

11. The fungus of claim 10, wherein the selectable marker is a drug selection marker.

12. The fungus of claim 1, wherein the CAD enzyme is an Aspergillus terreus CAD enzyme (Gene ID AB326105).

13. The fungus of claim 1, wherein the fungus has a Y. lipolytica PO1f genetic background.

14. The fungus of claim 1, wherein the second genetic modification comprises a transgenic nucleic acid molecule encoding an iron-regulatory protein operably linked to a promoter functional in the fungus.

15. The fungus of claim 14, wherein the iron-regulatory protein is a O. cuniculus iron-regulatory protein (Gen ID Q01059).

16. The fungus of claim 15, wherein the iron-regulatory protein comprises a S711D mutation relative to the wild type protein.

17. The fungus of claim 1, wherein the second genetic modification comprises a transgenic nucleic acid molecule encoding a small acid resistance transporter protein operably linked to a promoter functional in the fungus.

18. The fungus of claim 17, wherein the small acid resistance transporter is a Y. lipolytica small acid resistance transporter (Gen ID YALI0E10483g).

19. The fungus of claim 1, wherein the second genetic modification comprises a transgenic nucleic acid molecule encoding a citrate transport protein operably linked to a promoter functional in the fungus.

20. The fungus of claim 19, wherein the citrate transport protein is a Y. lipolytica citrate transport protein (Gen ID YALI0F26323g).

21. The fungus of claim 1, wherein the second genetic modification comprises a transgenic nucleic acid molecule encoding an aconitase operably linked to a promoter functional in the fungus.

22. The fungus of claim 19, wherein the aconitase is a Y. lipolytica aconitase (Gen ID YALI0D09361g).

23. The fungus of claim 21, wherein the aconitase does not include a mitochondrial localization signal (MLS).

24. The fungus of claim 21, further comprising a genome integrated and an episomal nucleic acid molecule each encoding a CAD enzyme operably linked to a promoter functional in the fungus.

25. The fungus of claim 1, wherein the second genetic modification comprises a transgenic nucleic acid molecule encoding citrate synthase operably linked to a promoter functional in the fungus.

26. The fungus of claim 19, wherein the citrate synthase is a Y. lipolytica citrate synthase (Gen ID YALI0E02684g).

27. The fungus of claim 25, wherein the citrate synthase does not include a MLS.

28. The fungus of claim 1, wherein the second genetic modification comprises a transgenic nucleic acid molecule encoding a phosphofructokinase enzyme operably linked to a promoter functional in the fungus.

29. The fungus of claim 28, wherein the phosphofructokinase is a Y. lipolytica phosphofructokinase (Gen ID YALI0D16357g).

30. The fungus of claim 29, wherein the phosphofructokinase comprises a K731A or K731R mutation relative to the wild type protein.

31. The fungus of claim 21, wherein the second genetic modification comprises a transgenic nucleic acid molecule encoding an AMPD enzyme operably linked to a promoter functional in the fungus.

32. The fungus of claim 31, wherein the AMPD enzyme is a Y. lipolytica AMPD enzyme (Gene ID YALI0E11495g).

33. The fungus of claim 31, wherein the transgenic nucleic acid molecule encoding a AMPD enzyme is integrated in the Y. lipolytica genome.

34. The fungus of claim 31, wherein the nucleic acid molecule encoding the AMPD enzyme is comprises in an UAS1B16-TEF expression cassette.

35. The fungus of claim 1, wherein the fungus has been adapted to low pH growth conditions.

36. A culture system comprising a population of transgenic oleaginous fungi in accordance with anyone of claims 1-35 and a growth medium.

37. The culture system of claim 36, wherein the culture produces itaconic acid.

38. The culture system of claim 36, wherein the medium comprises carbon and nitrogen sources, said carbon and nitrogen sources present in a molar ratio of at least 30 (C:N).

39. The culture system of claim 38, wherein said carbon and nitrogen sources are present in a ratio of between about 100 to 1,000 (C:N).

40. The culture system of claim 36, wherein the medium is not supplemented with amino acids.

41. The culture system of claim 36, comprised in a bioreactor.

42. A method for producing an organic commodity chemical comprising:
(a) culturing transgenic oleaginous fungi in accordance with any one of claims 1-35 in a growth media; and
(b) collecting the organic commodity chemical from the fungus and/or the growth media.

43. The method of claim 42, wherein the commodity chemical comprises itaconic acid.

44. The method of claim 42, wherein the culturing is in a bioreactor.

45. The method of claim 44, wherein the transgenic oleaginous fungi is a fungi in accordance with claim 24.

46. The method of claim 42, wherein the culturing is in a batch system.

47. The method of claim 42, wherein the culturing is in a fed-batch system.

48. The method of claim 42, wherein the culturing is in continuous feed system.


SEQUENCE LISTING (47 sequences)

SEQ ID NO: 1 - DNA, 35 bp, Artificial sequence
  Synthetic primer
ggaacggtagatctcgagcgtcccaaaaccttctc

SEQ ID NO: 2 - DNA, 34 bp, Artificial sequence
  Synthetic primer
gtggacgggccggcgtttggcgcccgttttttcg

SEQ ID NO: 3 - DNA, 30 bp, Artificial sequence
  Synthetic primer
gtattgattgtaattctgtaaatctatttc

SEQ ID NO: 4 - DNA, 60 bp, Artificial sequence
  Synthetic primer
cttgctgcaaagaccgcaggaaggacaatgcttgcagagtgtaggggggcttcgctgtgg

SEQ ID NO: 5 - DNA, 60 bp, Artificial sequence
  Synthetic primer
tttcatacaggctacggagcttgacgactaccacagcgaagcccccctacactctgcaag

SEQ ID NO: 6 - DNA, 20 bp, Artificial sequence
  Synthetic primer
gaggctctctgccgttgccc

SEQ ID NO: 7 - DNA, 20 bp, Artificial sequence
  Synthetic primer
ttcttgggggactgttggcc

SEQ ID NO: 8 - DNA, 27 bp, Artificial sequence
  Synthetic primer
agatgaagtaaccttcctggccagatc

SEQ ID NO: 9 - DNA, 20 bp, Artificial sequence
  Synthetic primer
ccgtccagctggtcgaccag

SEQ ID NO: 10 - DNA, 49 bp, Artificial sequence
  Synthetic primer
ctccttccttttcggttagagcggatgtggggggagggcgtgaatgtaa

SEQ ID NO: 11 - DNA, 33 bp, Artificial sequence
  Synthetic primer
gagtggcgcgccatgatttctgctattcgtccc

SEQ ID NO: 12 - DNA, 32 bp, Artificial sequence
  Synthetic primer
gcacttaattaattagagcttgaggccaacga

SEQ ID NO: 13 - DNA, 33 bp, Artificial sequence
  Synthetic primer
gagtggcgcgccatgcttaaggagcgattcgcc

SEQ ID NO: 14 - DNA, 32 bp, Artificial sequence
  Synthetic primer
gagtggcgcgccatgctggcttctcgagtttc

SEQ ID NO: 15 - DNA, 31 bp, Artificial sequence
  Synthetic primer
gcacttaattaattatttcttggaggcagcc

SEQ ID NO: 16 - DNA, 36 bp, Artificial sequence
  Synthetic primer
gagtggcgcgccatggccaacaacttcctcaacttc

SEQ ID NO: 17 - DNA, 31 bp, Artificial sequence
  Synthetic primer
gagtggcgcgccatgaccaaacaatctgcgg

SEQ ID NO: 18 - DNA, 32 bp, Artificial sequence
  Synthetic primer
gcacttaattaattataccagtggcgatttca

SEQ ID NO: 19 - DNA, 37 bp, Artificial sequence
  Synthetic primer
gagtggcgcgccatgtctaatccttttgcatacttag

SEQ ID NO: 20 - DNA, 35 bp, Artificial sequence
  Synthetic primer
gcacttaattaactactttgccatttttctaatca

SEQ ID NO: 21 - DNA, 24 bp, Artificial sequence
  Synthetic primer
aactctagatatgtctgataaaag

SEQ ID NO: 22 - DNA, 27 bp, Artificial sequence
  Synthetic primer
ttagcggccgcatactactgtatattc

SEQ ID NO: 23 - DNA, 27 bp, Artificial sequence
  Synthetic primer
aacgcggccgcctgcagactaaattta

SEQ ID NO: 24 - DNA, 25 bp, Artificial sequence
  Synthetic primer
ttcagatctctaacagttaatcttc

SEQ ID NO: 25 - DNA, 36 bp, Artificial sequence
  Synthetic primer
actgggcgcgccatgattgaaggaatctcctttgcg

SEQ ID NO: 26 - DNA, 39 bp, Artificial sequence
  Synthetic primer
actgttaattaactaacaaggatcaataataccctgctc

SEQ ID NO: 27 - DNA, 27 bp, Artificial sequence
  Synthetic primer
acgtgctcgcgacgtctgcttctgcca

SEQ ID NO: 28 - DNA, 27 bp, Artificial sequence
  Synthetic primer
tggcagaagcagacgtcgcgagcacgt

SEQ ID NO: 29 - DNA, 28 bp, Artificial sequence
  Synthetic primer
gacgtgctccggacgtctgcttctgcca

SEQ ID NO: 30 - DNA, 28 bp, Artificial sequence
  Synthetic primer
tggcagaagcagacgtccggagcacgtc

SEQ ID NO: 31 - DNA, 33 bp, Artificial sequence
  Synthetic primer
gactggcgcgccatgagagcccttctgaacaag

SEQ ID NO: 32 - DNA, 33 bp, Artificial sequence
  Synthetic primer
gtccttaattaatcatctcatcattcgtcggac

SEQ ID NO: 33 - DNA, 20 bp, Artificial sequence
  Synthetic primer
gccgaaccttggaagtccct

SEQ ID NO: 34 - DNA, 45 bp, Artificial sequence
  Synthetic primer
gtccttaattaactaaagaatctccatgatcttctcatagatggt

SEQ ID NO: 35 - DNA, 56 bp, Artificial sequence
  Synthetic primer
gactggcgcgccatggtttcatcagataccaagaaggccgaaccttggaagtccct

SEQ ID NO: 36 - protein, 490 aa, A. terreus
MTKQSADSNAKSGVTSEICHWASNLATDDIPSDVLERAKYLILDGIACAWVGARVPWSEKYVQATMSFEPPGACRVIGYGQKLGPVAAAMTNSAFIQATELDDYHSEAPLHSASIVLPAVFAASEVLAEQGKTISGIDVILAAIVGFESGPRIGKAIYGSDLLNNGWHCGAVYGAPAGALATGKLLGLTPDSMEDALGIACTQACGLMSAQYGGMVKRVQHGFAARNGLLGGLLAHGGYEAMKGVLERSYGGFLKMFTKGNGREPPYKEEEVVAGLGSFWHTFTIRIKLYACCGLVHGPVEAIENLQGRYPELLNRANLSNIRHVHVQLSTASNSHCGWIPEERPISSIAGQMSVAYILAVQLVDQQCLLSQFSEFDDNLERPEVWDLARKVTSSQSEEFDQDGNCLSAGRVRIEFNDGSSITESVEKPLGVKEPMPNERILHKYRTLAGSVTDESRVKEIEDLVLGLDRLTDISPLLELLNCPVKSPLV

SEQ ID NO: 37 - protein, 869 aa, Y. lipolytica
MPQQAMDIKGKAKSVPMPEEDDLDSHFVGPISPRPHGADEIAGYVGCEDDEDELEELGMLGRSASTHFSYAEERHLIEVDAKYRALHGHLPHQHSQSPVSRSSSFVRAEMNHPPPPPSSHTHQQPEDDDASSTRSRSSSRASGRKFNRNRTKSGSSLSKGLQQLNMTGSLEEEPYESDDDARLSAEDDIVYDATQKDTCKPISPTLKRTRTKDDMKNMSINDVKITTTTEDPLVAQELSMMFEKVQYCRDLRDKYQTVSLQKDGDNPKDDKTHWKIYPEPPPPSWHETEKRFRGSSKKEHQKKDPTMDEFKFEDCEIPGPNDMVFKRDPTCVYQVYEDESSLNENKPFVAIPSIRDYYMDLEDLIVASSDGPAKSFAFRRLQYLEAKWNLYYLLNEYTETTESKTNPHRDFYNVRKVDTHVHHSACMNQKHLLRFIKYKMKNCPDEVVIHRDGRELTLSQVFESLNLTAYDLSIDTLDMHAHKDSFHRFDKFNLKYNPVGESRLREIFLKTDNYIQGRYLAEITKEVFQDLENSKYQMAEYRISIYGRSKDEWDKLAAWVLDNKLFSPNVRWLIQVPRLYDIYKKAGLVNTFADIVQNVFEPLFEVTKDPSTHPKLHVFLQRVVGFDSVDDESKLDRRFHRKFPTAAYWDSAQNPPYSYWQYYLYANMASINTWRQRLGYNTFELRPHAGEAGDPEHLLCTYLVAQGINHGILLRKVPFIQYLYYLDQIPIAMSPVSNNALFLTFDKNPFYSYFKRGLNVSLSSDDPLQFAYTKEALIEEYSVAALIYKLSNVDMCELARNSVLQSGFERIIKEHWIGENYEIHGPEGNTIQKTNVPNVRLAFRDETLTHELALVDKYTNLEEFERLHG

SEQ ID NO: 38 - protein, 889 aa, O. cuniculus
MSNPFAYLAEPLDPAQPGKKFFNLNKLDYSRYGRLPFSIRVLLEAAVRNCDKFLVKKEDIENILNWNVTQHMNIEVPFKPARVILQDFTGVPSVVDFAAMRDAVKKLGGDPEKINPICPVDLVIDHSIQVDFNRRADSLQKNQDLEFERNRERFEFLKWGSKAFRNMRIIPPGSGIIHQVNLEYLARVVFDQDGYYYPDSLVGTDSHTTMIDGLGVLGWGVGGIEAEAVMLGQPISMVLPQVIGYRLMGKPHPLVTSTDIVLTITKHLRQVGVVGKFVEFFGLGVAQLSIADRATIANMCPEYGATATFFPVDEVSIKYLVQTGRDESKVKQIRKYLQAVGMFRDYSDPSQDPDFTQVVELDLKTVVPCCSGPKRPQDKVAVSDMKKDFESCLGAKQGFKGFQVAPDHHNDHKTFIYNDSEFTLSHGSVVIAAITSCTNTSNPSVMLGAGLLAKKAVDAGLNVKPYVKTSLSPGSGVVTYYLRESGVMPYLSQLGFDVVGYGCMTCIGNSGPLPEPVVEAITQGDLVAVGVLSGNRNFEGRVHPNTRANYLASPPLVIAYAIAGTIRIDFEKEPLGTNAKGQQVFLRDIWPTREEIQAVERQYVIPGMFTEVYQKIETVNASWNALAAPSDKLYLWNPKSTYIKSPPFFENLTLDLQPPKSIVDAYVLLNLGDSVTTDHISPAGNIARNSPAARYLTNRGLTPREFNSYGSRRGNDAIMARGTFANIRLLNRFLNKQAPQTIHLPSGETLDVFDAAERYQQEGHPLIVLAGKEYGSGSSRDWAAKGPFLLGIKAVLAESYERIHRSNLVGMGVIPLEYLPGENADSLGLTGRERYTIIIPENLTPRMHVQVKLDTGKTFQAVIRFDTDVELTYLHNGGILNYMIRKMAK

SEQ ID NO: 39 - protein, 779 aa, Y. lipolytica
MLASRVSIKAPRLARSLATTTNASLNLDSKVRMNNWEANNFLNFKKHTENVQIVKERLNRPLTYAEKILYGHLDKPHEQEIVRGQSYLKLRPDRAACQDATAQMAILQFMSAGIPTVQTPTTVHCDHLIQAQVGGEQDLARAIDINKEVYNFLGTASAKYDIGFWKAGSGIIHQIILENYAFPGALLIGSDSHTPNAGGLGMLAIGVGGADVVDVMAGLPWELKAPKIIGVKLTGKLSGWTSPKDIILKVAGILTVKGGTGAIVEYFGDGVDNLSCTGMGTICNMGAEIGATTSTFPFNERMADYLNATGRKEIADFARLYNHFLSADEGCEYDQLIEIDLNTLEPYVNGPFTPDLATPISKLKDVAVENGWPLEVKVGLIGSCTNSSYEDMERSASIAKDAMAHGLKSKSIYTVTPGSEQIRATIERDGQLQTFLDFGGIVLANACGPCIGQWDRRDIKKGEKNTIVSSYNRNFTGRNDSNPATHAFVTSPDLVTAFAIAGDLRFNPLTDSLKDSEGKEFKLKEPTGKGLPDRGYDPGMDTYQAPPADRSAVEVDVSPTSDRLQILKPFKPWDGKDGIDMPILIKSLGKTTTDHISQAGPWLKYRGHLQNISNNYMIGAINAENEEANNVRNQITGEWGGVPETAIAYRDNGIRWVVVGGDNFGEGSSREHAALEPRFLGGFAIITKSFARIHETNLKKQGLLPLNFVNGADYDKIQPSDKISILGLKDLAPGKNVTIEVTPKDGAKWTTEVSHTYNSEQLEWFKYGSALNKMAASKK

SEQ ID NO: 40 - protein, 743 aa, Y. lipolytica
MANNFLNFKKHTENVQIVKERLNRPLTYAEKILYGHLDKPHEQEIVRGQSYLKLRPDRAACQDATAQMAILQFMSAGIPTVQTPTTVHCDHLIQAQVGGEQDLARAIDINKEVYNFLGTASAKYDIGFWKAGSGIIHQIILENYAFPGALLIGSDSHTPNAGGLGMLAIGVGGADVVDVMAGLPWELKAPKIIGVKLTGKLSGWTSPKDIILKVAGILTVKGGTGAIVEYFGDGVDNLSCTGMGTICNMGAEIGATTSTFPFNERMADYLNATGRKEIADFARLYNHFLSADEGCEYDQLIEIDLNTLEPYVNGPFTPDLATPISKLKDVAVENGWPLEVKVGLIGSCTNSSYEDMERSASIAKDAMAHGLKSKSIYTVTPGSEQIRATIERDGQLQTFLDFGGIVLANACGPCIGQWDRRDIKKGEKNTIVSSYNRNFTGRNDSNPATHAFVTSPDLVTAFAIAGDLRFNPLTDSLKDSEGKEFKLKEPTGKGLPDRGYDPGMDTYQAPPADRSAVEVDVSPTSDRLQILKPFKPWDGKDGIDMPILIKSLGKTTTDHISQAGPWLKYRGHLQNISNNYMIGAINAENEEANNVRNQITGEWGGVPETAIAYRDNGIRWVVVGGDNFGEGSSREHAALEPRFLGGFAIITKSFARIHETNLKKQGLLPLNFVNGADYDKIQPSDKISILGLKDLAPGKNVTIEVTPKDGAKWTTEVSHTYNSEQLEWFKYGSALNKMAASKK

SEQ ID NO: 41 - protein, 465 aa, Y. lipolytica
MISAIRPAVRSSVRVAPMANTAFRAYSTQDGLKERFAELIPENVEKIKKLRKEKGNTVIGEVILDQAYGGMRGIKGLVWEGSVLDPEEGIRFRGLTIPDLQKQLPHAPGGKEPLPEGLFWLLLTGEIPTDAQVKGLSADWASRAEIPKHVEELIDRCPPTLHPMAQLGIAVNALESESQFTKAYEKGVNKKEYWQYTYEDSMNLIAKLPVIASRIYRNLFKDGKIVGSIDNSLDYSANFASLLGFGDNKEFIELLRLYLTIHADHEGGNVSAHTTKLVGSALSSPFLSLSAGLNGLAGPLHGRANQEVLEWILEMKSKIGSDVTKEDIEKYLWDTLKAGRVVPGYGHAVLRKTDPRYTAQREFALEHMPDYDLFHLVSTIYEVAPKVLTEHGKTKNPWPNVDSHSGVLLQYYGLTEQSYYTVLFGVSRAIGVLPQLIMDRAYGAPIERPKSFSTEKYAELVGLKL

SEQ ID NO: 42 - protein, 436 aa, Y. lipolytica
MGLKERFAELIPENVEKIKKLRKEKGNTVIGEVILDQAYGGMRGIKGLVWEGSVLDPEEGIRFRGLTIPDLQKQLPHAPGGKEPLPEGLFWLLLTGEIPTDAQVKGLSADWASRAEIPKHVEELIDRCPPTLHPMAQLGIAVNALESESQFTKAYEKGVNKKEYWQYTYEDSMNLIAKLPVIASRIYRNLFKDGKIVGSIDNSLDYSANFASLLGFGDNKEFIELLRLYLTIHADHEGGNVSAHTTKLVGSALSSPFLSLSAGLNGLAGPLHGRANQEVLEWILEMKSKIGSDVTKEDIEKYLWDTLKAGRVVPGYGHAVLRKTDPRYTAQREFALEHMPDYDLFHLVSTIYEVAPKVLTEHGKTKNPWPNVDSHSGVLLQYYGLTEQSYYTVLFGVSRAIGVLPQLIMDRAYGAPIERPKSFSTEKYAELVGLKL

SEQ ID NO: 43 - protein, 516 aa, Y. lipolytica
MRALLNKADETFASTGGSIDIELDSIDQKLPRVSVSSDLGSGSDTAGEGDGPVSANTDDSNTNTLDVEALPATAPDDDVKFNRFTLSQKRIMTAVLAFCFFQPFLVTYAMLPAVPLIAEQFDVSGTIVTVGNAIFFLITGFSSCFFGPFSDAYGRKAALITCCIIFIVSNIGIAASPNLVSYYIFRATTAMGGTAFFSVSGSAIADIWRPEHRGKAVGACLLGSQSGMTIGPIIGGWIVTKTSWRVIFWMQAGVALANLLLVTFVLREPMATTKHQMLCAEQNKRFVWIWINPFKVLMGLRNMHLLLAGLSIVPIMYGMDCLLTPLSLVVEPRFDIKSPVVAALFYLPQGVGFLIGCYFGGMYADKTVQRWTKIRGRRVCEDRLRSQLPFVGILLPVCMLIYGWSLEKEFGGVAVPVVTMFLVGFGLSMYFPSLNAYCADSSPELGTAAAISGNYAIRNCGSAIAAASTLKAVENIGIGWTSTVAAFGFIASTIPVFILLWKGEDMRQKAVRRMMR

SEQ ID NO: 44 - protein, 292 aa, Y. lipolytica
MVSSDTKKAEPWKSLVAGSTAGAVEGLVTYPFEWSKTRLQLVDKSSTASRNPLVLIYNTAKTQGLGAVYTGCPAFIVGNTVKAGVRFLGFDAIKGLLADKDGKVSGPRGVLAGLGAGVLESVVAVTPFETIKTAMIDDRQSKNPKYQGLFKGTAQLIKDKGLSGIYRGLVPVTMRQAANQAVRLGSYNWMKVFIQSRQKDPKAPLSSLSTFIVGAFAGIVTVYTTMPLDTVKTRMQSLEAKKEYRGTFHCFARIFKEEGLLTFWKGATPRLGRLILSGGIVFTIYEKIMEIL

SEQ ID NO: 45 - protein, 953 aa, Y. lipolytica
MIEGISFASFVTHEKPKFVRALDFYKALGFLPTKEYKHGTDHHATDEEGAGSIQEVWLTSSRAGVPSVTVKLRLSRHGNEHVSLPNLKHDWRSLVPSLVYYAPDLDAVRAAITPFLHEDHSTLLERPSHTNFIELYAIDPMGNLVGFSRRENPYSSAMQKPFSADDIGPQNFSKPNETKIKGKKRIGVMTSGGDAPGMCAAVRAVVRAGIARGCEVYAVREGYEGLVKGGDLIEPLSWEDVRGWLSLGGTLIGTARCKEFREREGRLAGALNMVKNGIDALIVIGGDGSLTGADLFREEWPSLIEELVTNGSITAEQAERHRHLDICGMVGSIDNDMATTDVTIGAYSSLDRICELVDFIDATAQSHSRAFVVEVMGRHCGWLALMAGTATGADYIFIPEAAPDATQWAEKMTRVVKRHRSQGKRKTVVIVAEGAIDSDLNPITAKMVKDVLDGIGLDTRISTLGHVQRGGPPVAADRVLASLQGVEAIDAILSLTPETPSPMIALNENKITRKPLVESVALTKKVADAIGNKDFAEAMRLRNPEFVEQLQGFLLTNSADKDRPQEPAKDPLRVAIVCTGAPAGGMNAAIRSAVLYGLARGHQMFAIHNGWSGLVKNGDDAVRELTWLEVEPLCQKGGCEIGTNRSLPECDLGMIAYHFQRQRFDGLIVIGGFEAFRALNQLDDARHAYPALRIPMVGIPATISNNVPGTDYSLGADTCLNSLVQYCDVLKTSASATRLRLFVVEVQGGNSGYIATVAGLITGAYVVYTPESGINLRLLQHDISYLKDTFAHQADVNRTGKLLLRNERSSNVFTTDVITGIINEEAKGSFDARTAIPGHVQQGGHPSPTDRVRAQRFAIKAVQFIEEHHGSKNNADHCVILGVRGSKFKYTSVSHLYAHKTEHGARRPKHSYWHAIGDIANMLVGRKAPPLPETLNDEIEKNIAKEQGIIDPC

SEQ ID NO: 46 - protein, 953 aa, Y. lipolytica
MIEGISFASFVTHEKPKFVRALDFYKALGFLPTKEYKHGTDHHATDEEGAGSIQEVWLTSSRAGVPSVTVKLRLSRHGNEHVSLPNLKHDWRSLVPSLVYYAPDLDAVRAAITPFLHEDHSTLLERPSHTNFIELYAIDPMGNLVGFSRRENPYSSAMQKPFSADDIGPQNFSKPNETKIKGKKRIGVMTSGGDAPGMCAAVRAVVRAGIARGCEVYAVREGYEGLVKGGDLIEPLSWEDVRGWLSLGGTLIGTARCKEFREREGRLAGALNMVKNGIDALIVIGGDGSLTGADLFREEWPSLIEELVTNGSITAEQAERHRHLDICGMVGSIDNDMATTDVTIGAYSSLDRICELVDFIDATAQSHSRAFVVEVMGRHCGWLALMAGTATGADYIFIPEAAPDATQWAEKMTRVVKRHRSQGKRKTVVIVAEGAIDSDLNPITAKMVKDVLDGIGLDTRISTLGHVQRGGPPVAADRVLASLQGVEAIDAILSLTPETPSPMIALNENKITRKPLVESVALTKKVADAIGNKDFAEAMRLRNPEFVEQLQGFLLTNSADKDRPQEPAKDPLRVAIVCTGAPAGGMNAAIRSAVLYGLARGHQMFAIHNGWSGLVKNGDDAVRELTWLEVEPLCQKGGCEIGTNRSLPECDLGMIAYHFQRQRFDGLIVIGGFEAFRALNQLDDARHAYPALRIPMVGIPATISNNVPGTDYSLGADTCLNSLVQYCDVLATSASATRLRLFVVEVQGGNSGYIATVAGLITGAYVVYTPESGINLRLLQHDISYLKDTFAHQADVNRTGKLLLRNERSSNVFTTDVITGIINEEAKGSFDARTAIPGHVQQGGHPSPTDRVRAQRFAIKAVQFIEEHHGSKNNADHCVILGVRGSKFKYTSVSHLYAHKTEHGARRPKHSYWHAIGDIANMLVGRKAPPLPETLNDEIEKNIAKEQGIIDPC

SEQ ID NO: 47 - protein, 953 aa, Y. lipolytica
MIEGISFASFVTHEKPKFVRALDFYKALGFLPTKEYKHGTDHHATDEEGAGSIQEVWLTSSRAGVPSVTVKLRLSRHGNEHVSLPNLKHDWRSLVPSLVYYAPDLDAVRAAITPFLHEDHSTLLERPSHTNFIELYAIDPMGNLVGFSRRENPYSSAMQKPFSADDIGPQNFSKPNETKIKGKKRIGVMTSGGDAPGMCAAVRAVVRAGIARGCEVYAVREGYEGLVKGGDLIEPLSWEDVRGWLSLGGTLIGTARCKEFREREGRLAGALNMVKNGIDALIVIGGDGSLTGADLFREEWPSLIEELVTNGSITAEQAERHRHLDICGMVGSIDNDMATTDVTIGAYSSLDRICELVDFIDATAQSHSRAFVVEVMGRHCGWLALMAGTATGADYIFIPEAAPDATQWAEKMTRVVKRHRSQGKRKTVVIVAEGAIDSDLNPITAKMVKDVLDGIGLDTRISTLGHVQRGGPPVAADRVLASLQGVEAIDAILSLTPETPSPMIALNENKITRKPLVESVALTKKVADAIGNKDFAEAMRLRNPEFVEQLQGFLLTNSADKDRPQEPAKDPLRVAIVCTGAPAGGMNAAIRSAVLYGLARGHQMFAIHNGWSGLVKNGDDAVRELTWLEVEPLCQKGGCEIGTNRSLPECDLGMIAYHFQRQRFDGLIVIGGFEAFRALNQLDDARHAYPALRIPMVGIPATISNNVPGTDYSLGADTCLNSLVQYCDVLRTSASATRLRLFVVEVQGGNSGYIATVAGLITGAYVVYTPESGINLRLLQHDISYLKDTFAHQADVNRTGKLLLRNERSSNVFTTDVITGIINEEAKGSFDARTAIPGHVQQGGHPSPTDRVRAQRFAIKAVQFIEEHHGSKNNADHCVILGVRGSKFKYTSVSHLYAHKTEHGARRPKHSYWHAIGDIANMLVGRKAPPLPETLNDEIEKNIAKEQGIIDPC